(19) United States
(12) Patent Application Publication
(10) Pub. No.: US 2024/0366807 A1
(43) Pub. Date: Nov. 7, 2024

(54) RADIOFLUORINATED AGENTS FOR PET IMAGING SELECTIVELY TARGETING FIBROBLAST ACTIVATION PROTEIN

(71) Applicant: Trustees of tufts college, Medford, MA (US)

(72) Inventors: William W. Bachovchin, Cambridge, MA (US); Hung-sen Lai, Andover, MA (US); Wengen Wu, Winchester, MA (US)

(21) Appl. No.: 18/683,663

(22) PCT Filed: Aug. 18, 2022
(86) PCT No.: PCT/US22/40778

§ 371 Date: Feb. 14, 2024

Related U.S. Application Data
(60) Provisional application No. 63/234,435, filed on Aug. 18, 2021.

(51) Int. Cl. A61K 51/04; A61P 35/00; C07B 59/00; C12N 9/99

(52) U.S. Cl. CPC A61K 51/0455; A61K 51/0446; A61P 35/00; C07B 59/004; C12N 9/99; A61K 2121/00; A61K 2123/00; C07B 2200/05

(57) ABSTRACT

Disclosed are compounds useful for imaging cells that overexpress FAP, and for treating cancer. Also disclosed are methods of making said compounds.

### CROSS-REFERENCE TO RELATED APPLICATION

This application claims the benefit of priority of U.S. Provisional Application No. 63/234,435, filed on Aug. 18, 2021; which is incorporated herein by reference in its entirety.

### BACKGROUND

Fibroblast-activation protein (FAP), also known as seprase, is a type II integral membrane serine peptidase. FAP belongs to the dipeptidyl peptidase IV family. It is a 170 kDa homodimer containing two N-glycosylated subunits with a large C-terminal extracellular domain, in which the enzyme's catalytic domain is located. FAP, in its glycosylated form, has both post-prolyl dipeptidyl peptidase and gelatinase activities. Homologues of human FAP were found in several species, including mice and cynomolgus monkeys.

FAP is expressed selectively in reactive stromal fibroblasts of more than 90% of epithelial malignancies (primary and metastatic) examined, including lung, colorectal, bladder, ovarian and breast carcinomas, and in malignant mesenchymal cells of bone and soft tissue sarcomas, while it is generally absent from normal adult tissues (Brennen et al., Mol. Cancer Ther. 11 (2): 257-266 (2012); Garin-Chesa et al., Proc Natl Acad Sci USA 87, 7235-7239 (1990); Rettig et al., Cancer Res. 53:3327-3335 (1993); Rettig et al., Proc Natl Acad Sci USA 85, 3110-3114 (1988)). FAP is also expressed on certain malignant tumor cells.

Due to its expression in many common cancers and its restricted expression in normal tissues, FAP is a promising antigenic target for imaging, diagnosis, and therapy of a variety of cancers. Accordingly, there is an ongoing need for therapies that target FAP.

### SUMMARY OF THE INVENTION

In one aspect, the present disclosure provides a compound having a structure represented by formula I, or a pharmaceutically acceptable salt thereof:

Z-A-(FAPx)ₙ₁ (I)

wherein,
FAPx is FAPi or FAPb;
FAPi is a compound that covalently binds to FAP;
FAPb is a compound that non-covalently binds to FAP;
A is cycloalkyl, aryl, heteroaryl, or heterocyclyl;
Z is a radioactive halogen isotope or trialkylammonium; and
n1 is 1-6.

In one aspect, the present disclosure provides a compound having a structure represented by formula XI, or a pharmaceutically acceptable salt thereof:

(Z)_{z1}-A₂-L₂-A₁-(FAPX)ₙ₁ (XI)

wherein,
FAPx is FAPi or FAPb;
FAPi is a compound which covalently binds to FAP;
FAPb is a compound which non-covalently binds to FAP;
each A₁, and A₂ is independently cycloalkyl, aryl, heteroaryl, or heterocyclyl;
L² is a bond or a covalent linker;
Z is a radioactive halogen isotope or trialkylammonium;
each z1 is independently 1-3; and
n1 is 1-6.

In one aspect, the present disclosure provides a compound having a structure represented by formula XXI, or a pharmaceutically acceptable salt thereof:

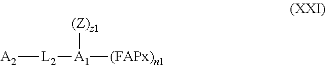

wherein,
FAPx is FAPi or FAPb;
FAPi is a compound which covalently binds to FAP;
FAPb is a compound which non-covalently binds to FAP;
each A₁, and A₂ is independently cycloalkyl, aryl, heteroaryl, or heterocyclyl;
L₂ is a bond or a covalent linker;
Z is a radioactive halogen isotope or trialkylammonium;
each z1 is independently 1-3; and
n1 is 1-6.

In further aspects, the present disclosure provides compositions, comprising a compound of formula I or a pharmaceutically acceptable salt thereof; diagnostic or therapeutic methods of using a compound of formula I or a pharmaceutically acceptable salt thereof; kits comprising a compound of formula I or a pharmaceutically acceptable salt thereof, and methods of making a compound of formula I or a pharmaceutically acceptable salt thereof.

More particularly, pharmaceutical compositions are provided including at least one compound as disclosed herein, including a compound of any of the formulae disclosed herein (including formulae I, XI, XXI, II, XII, XXII, III, XIII, XXIII, IV-A, IV-B, IV-C, IV-D, IX-E, IX-F, XIV-A, XXIV-A, XIV-B, XXIV-B, XIV-C, and/or XXIV-C), and, optionally, a pharmaceutically acceptable carrier and/or excipient. In certain embodiments, the pharmaceutical composition is intended for use in the diagnosis or treatment of a disease characterized by overexpression of fibroblast activation protein (FAP) in an animal, preferably a human subject.

In yet another aspect, kits are provided comprising or consisting of least one compound as disclosed herein including a compound of any of the formulae disclosed herein (including formulae I, XI, XXI, II, XII, XXII, III, XIII, XXIII, IV-A, IV-B, IV-C, IV-D, IX-E, IX-F, XIV-A, XXIV-A, XIV-B, XXIV-B, XIV-C and/or XXIV-C), and instructions for the diagnosis or treatment of a disease.

In yet an additional aspect, methods are provided for diagnosing, imaging or reducing tissue overexpressing FAP in an animal (preferably a human patient), comprising administering to the animal least one compound as disclosed herein including a compound of any of the formulae disclosed herein (including formulae I, XI, XXI, II, XII, XXII, III, XIII, XXIII, IV-A, IV-B, IV-C, IV-D, IX-E, IX-F, XIV-A, XXIV-A, XIV-B, XXIV-B, XIV-C and/or XXIV-C).

Methods for treating a subject suffering from a tumor or cancer are also provided which may comprise administering to a subject in need thereof an effective amount least one compound as disclosed herein including a compound of any of the formulae disclosed herein (including formulae I, XI, XXI, IL XII, XXII, III, XIII, XXIII, IV-A, IV-B, IV-C, IV-D, IX-E, IX-F, XIV-A, XXIV-A, XIV-B, XXIV-B, XIV-C and/or XXIV-C). Subjects for treatment may include a human patient diagnosed with cancer, such as a tumor (e.g. solid tumor), including subjects diagnosed and selected for treatment of prostate cancer.

### DETAILED DESCRIPTION OF THE INVENTION

The tumor stroma accounts for a large part of the tumor mass. Among the stroma is a subpopulation of cells known as cancer-associated fibroblasts (CAFs), which are present in more than 90% of epithelial carcinomas, including pancreatic cancer, colon cancer, and breast cancer. Cancer-associated fibroblasts feature high expression of FAP, which is not detectable in adult normal tissue but is associated with a poor prognosis in cancer patients. Thus, CAFs and FAP represent an attractive target for the delivery of diagnostic and therapeutic compounds.

Disclosed herein are agents and methods of use thereof for imaging FAP via methods such as ¹⁸F position emission tomography.

### Compounds

In one aspect, the present disclosure provides compounds having a structure represented by formula I or a pharmaceutically acceptable salt thereof:

Z-A-(FAPx)ₙ₁ (I)

wherein,
FAPx is FAPi or FAPb;
FAPi is a compound that covalently binds to FAP;
FAPb is a compound that non-covalently binds to FAP;
A is cycloalkyl, aryl, heteroaryl, or heterocyclyl;
Z is a radioactive halogen isotope or trialkylammonium; and
n1 is 1-6.

In an aspect, the present disclosure provides compounds having a structure represented by formula XI, or a pharmaceutically acceptable salt thereof:

(Z)_{z1}-A₂-L₂-A₁-(FAPx)ₙ₁ (XI)

wherein,
FAN is FAPi or FAPb;
FAPi is a compound which covalently binds to FAP;
FAPb is a compound which non-covalently binds to FAP;
each A₁, and A₂ is independently cycloalkyl, aryl, heteroaryl, or heterocyclyl;
L² is a bond or a covalent linker (e.g., a substituted or unsubstituted C₁-C₁₂ alkylene, or a substituted or unsubstituted 2 to 12 membered heteroalkylene);
Z is a radioactive halogen isotope or trialkylammonium;
each z1 is independently 1-3; and
n1 is 1-6.

In an aspect, the present disclosure provides compounds having a structure represented by formula XXI or a pharmaceutically acceptable salt thereof:

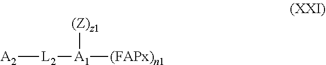

wherein,
FAPx is FAPi or FAPb;
FAPi is a compound which covalently binds to FAP;
FAPb is a compound which non-covalently binds to FAP;
each A, A₁, and A₂ is independently cycloalkyl, aryl, heteroaryl, or heterocyclyl;
L² is a bond or a covalent linker (e.g., a substituted or unsubstituted C₁-C₁₂ alkylene, or a substituted or unsubstituted 2 to 12 membered heteroalkylene);
Z is a radioactive halogen isotope or trialkylammonium;
each z1 is independently 1-3; and
n1 is 1-6.

In certain embodiments, n₁ is 1.

In certain embodiments, FAN is FAPi. In certain embodiments, FAPi covalently binds to a side chain of an amino acid in an active site of FAP. In certain embodiments, the covalent bond between FAPi and FAP is reversible. In certain embodiments, the covalent bond between FAPi and FAP is irreversible. In certain embodiments, FAPi comprises a moiety which has a Kᵢ for FAP that is at least 10× smaller as compared to the Kᵢ of prolyl endopeptidase for FAP (EC 3.4.21.26; PREP). In certain embodiments, FAPi comprises a moiety which has a K, for FAP that is at least 100× smaller as compared to the Kᵢ of prolyl endopeptidase for FAP (EC 3.4.21.26; PREP). In certain embodiments, FAPi comprises a moiety which has a Kᵢ for FAP that is at least 1,000× smaller as compared to the Kᵢ of prolyl endopeptidase for FAP (EC 3.4.21.26; PREP). In certain embodiments, FAPi comprises a moiety which has a Kᵢ for FAP that is at least 5,000× smaller as compared to the Kᵢ of prolyl endopeptidase for FAP (EC 3.4.21.26; PREP). In certain embodiments, FAPi comprises a moiety which has a Kᵢ for FAP that is at least 10,000× smaller as compared to the Kᵢ of prolyl endopeptidase for FAP (EC 3.4.21.26; PREP). In certain embodiments, FAPi comprises a moiety which has a Kᵢ for FAP that is less than 10′M. In certain embodiments, FAPi comprises a moiety which has a Kᵢ for FAP that is less than 10⁷M. In certain embodiments, FAPi comprises a moiety which has a Kᵢ for FAP that is less than 10⁻⁸M. In certain embodiments, FAPi comprises a moiety which has a Kᵢ for FAP that is less than 10⁻⁹M. In certain embodiments, FAPi comprises a moiety which has a Kᵢ for FAP that is less than 10⁻¹⁰M.

In certain embodiments, FAPx is FAPb. In certain embodiments, FAP_{b} forms a complex with FAP. In certain embodiments, FAP_{b} comprises a moiety which has a K_{d} for FAP that is less than 10⁻⁶M. In certain embodiments, FAP_{b} comprises a moiety which has a K_{d} for FAP that is less than 10⁻⁷M. In certain embodiments, FAP_{b} comprises a moiety which has a K_{d} for FAP that is less than 10⁻⁸M. In certain embodiments, FAP_{b} comprises a moiety which has a K_{d} for FAP that is less than 10⁻⁹M. In certain embodiments, FAP_{b} comprises a moiety which has a K_{d} for FAP that is less than 10⁻¹⁰M.

In certain embodiments, the compound has a structure represented by formula II or a pharmaceutically acceptable salt thereof:

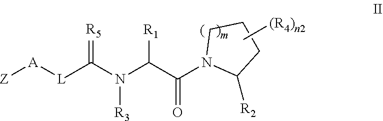

wherein,
A is cycloalkyl, aryl, heteroaryl, or heterocyclyl;
Z is a radioactive halogen isotope or a trialkylammonium;
L is a bond or a covalent linker (e.g., a substituted or unsubstituted C₁-C₁₂ alkylene, or a substituted or unsubstituted 2 to 12 membered heteroalkylene);
R₁ is H or alkyl;
R₂ is a moiety which covalently binds to a side chain of an amino acid in an active site of FAP;
R₃ is H or alkyl;
R₄ is alkyl, hydroxyl, amino, or halo;
R₅ is O or S;
m is 1-3; and
n2 is 0-7.

In certain embodiments, the compound has a structure represented by formula (XII), or a pharmaceutically acceptable salt thereof:

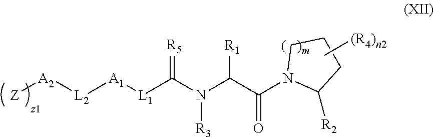

wherein,
each A&, and A₂ is independently is cycloalkyl, aryl, heteroaryl, or heterocyclyl;
Z is a radioactive halogen isotope or a trialkylammonium;
each L₁, and L² is independently a bond, a substituted or unsubstituted C₁-C₁₂ alkylene, or a substituted or unsubstituted 2 to 12 membered heteroalkylene;
R₁ is H or alkyl;
R₂ is a moiety which covalently binds to a side chain of an amino acid in an active site of FAP;
R₃ is H or alkyl;
R₄ is alkyl, hydroxyl, amino, or halo;
R₅ is O or S;
m is 1-3;
z1 is 1-3; and
n2 is 0-7.

In certain embodiments, the compound has a structure represented by formula (XXII), or a pharmaceutically acceptable salt thereof:

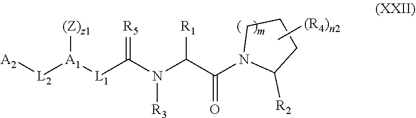

wherein,
each A₁, and A₂ is independently is cycloalkyl, aryl, heteroaryl, or heterocyclyl;
Z is a radioactive halogen isotope or a trialkylammonium;
each L₁, and L² is independently a bond, a substituted or unsubstituted C₁-C₁₂ alkylene, or a substituted or unsubstituted 2 to 12 membered heteroalkylene;
R₁ is H or alkyl;
R₂ is a moiety which covalently binds to a side chain of an amino acid in an active site of FAP;
R₃ is H or alkyl;
R₄ is alkyl, hydroxyl, amino, or halo;
R₅ is O or S;
m is 1-3;
z1 is 1-3; and
n2 is 0-7.

In certain embodiments, the compound has a structure represented by formula III or a pharmaceutically acceptable salt thereof:

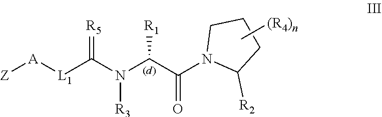

wherein,
A is cycloalkyl, aryl, heteroaryl, or heterocyclyl;
Z is a radioactive halogen isotope or a trialkylammonium;
L is a bond or a covalent linker (e.g., a substituted or unsubstituted C₁-C₁₂ alkylene, or a substituted or unsubstituted 2 to 12 membered heteroalkylene);
R₁ is H or alkyl;
R₂ is a moiety which covalently binds to a side chain of an amino acid in an active site of FAP;
R₃ is H or alkyl;
R₄ is alkyl, hydroxyl, amino, or halo;
R₅ is O or S; and
n is 0-7.

In certain embodiments, the compound has a structure represented by formula (XIII), or a pharmaceutically acceptable salt thereof:

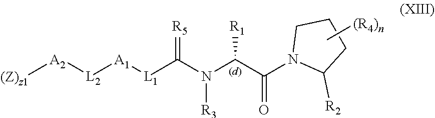

wherein,
each A₁, and A₂ is independently cycloalkyl, aryl, heteroaryl, or heterocyclyl;
Z is a radioactive halogen isotope or a trialkylammonium;
each L₁, and L² is independently a bond, a substituted or unsubstituted C₁-C₁₂ alkylene, or a substituted or unsubstituted 2 to 12 membered heteroalkylene;
R₁ is H or alkyl;
R₂ is a moiety which covalently binds to a side chain of an amino acid in an active site of FAP;
R₃ is H or alkyl;
R₄ is alkyl, hydroxyl, amino, or halo;
R₅ is O or S;
z1 is 1-3; and n is 0-7.

In certain embodiments, the compound has a structure represented by formula (XXIII), or a pharmaceutically acceptable salt thereof:

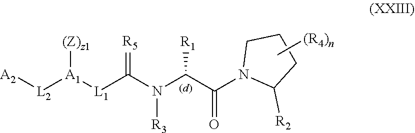

wherein,
each A₁, and A₂ is independently cycloalkyl, aryl, heteroaryl, or heterocyclyl;
Z is a radioactive halogen isotope or a trialkylammonium;
each L₁, and L² is independently a bond, a substituted or unsubstituted C₁-C₁₂ alkylene, or a substituted or unsubstituted 2 to 12 membered heteroalkylene;
R₁ is H or alkyl;
R₂ is a moiety which covalently binds to a side chain of an amino acid in an active site of FAP;
R₃ is H or alkyl;
R₄ is alkyl, hydroxyl, amino, or halo;
R₅ is O or S;
z1 is 1-3; and
n is 0-7.

In certain embodiments, R₁ is H. In certain embodiments, R₁ is H, or (C₁-C₆)alkyl (e.g., methyl or ethyl), which is optionally substituted with —OH. In certain embodiments, R¹ is —CH₃, or —CH₂—OH.

In certain embodiments, R₂ is B(Y¹)(Y²), CN, or formyl; wherein Y¹ and Y² are each hydroxyl; or Y¹ and Y² together with the boron atom to which they are attached combine to form a moiety which is hydrolysable to a boronic acid. In certain embodiments, Y¹ and Y² together with the boron atom to which they are attached combine to form a 5- to 8-membered ring. In certain embodiments, R₂ is B(OH)₂.

In certain embodiments, R₃ is (C₁-C₆)alkyl. In certain embodiments, R₃ is H.

In certain embodiments, R₄ is (C₁-C₆)alkyl. In certain embodiments, R₄ is halo (e.g., fluorine).

In certain embodiments, R₅ is O.

In certain embodiments, n or n2 is 2. In certain embodiments, n or n2 is 1. In certain embodiments, n or n₂ is 0.

In certain embodiments, L is a bond. In certain embodiments, L is a substituted or unsubstituted C₁-C₁₂ alkylene. In certain embodiments, L is a substituted or unsubstituted 2 to 12 membered heteroalkylene.

In certain embodiments, L¹ is a bond. In certain embodiments, L¹ is a substituted or unsubstituted C₁-C₁₂ alkylene. In certain embodiments, L¹ is a substituted or unsubstituted 2 to 12 membered heteroalkylene.

In certain embodiments, L² is a substituted or unsubstituted C₁-C₁₂ alkylene. In certain embodiments, L² is a substituted or unsubstituted 2 to 12 membered heteroalkylene. In certain embodiments, L² is an oxo-substituted or unsubstituted 2 to 12 membered heteroalkylene. In certain embodiments, L² is —(CH₂)_{q1}—C(O)—(NH)_{q2}—(CH₂)_{q3}—; and each q1, q2, and q3 is independently 0 to 5. In certain embodiments, L² is —(CH₂)_{q1}—C(O)(NH)_{q2}—(CH₂)_{q3}—C(O)(NH)_{q4}—(CH₂)_{q5}—; and each q1, q2, q3, q4, and q5 is independently 0 to 5.

In certain embodiments, q1 is 1. In certain embodiments, q1 is 2. In certain embodiments, q1 is 3. In certain embodiments, q1 is 4. In certain embodiments, q1 is 5.

In certain embodiments, q2 is 0. In certain embodiments, q2 is 1. In certain embodiments, q2 is 2.

In certain embodiments, q3 is 0. In certain embodiments, q3 is 1. In certain embodiments, q3 is 2.

In certain embodiments, q4 is 0. In certain embodiments, q4 is 1. In certain embodiments, q4 is 2.

In certain embodiments, q5 is 0. In certain embodiments, q5 is 1. In certain embodiments, q5 is 2.

In certain embodiments, L² is —(CH₂)_{q1}—C(O)—(NH)—(CH₂)_{q3}—. In certain embodiments, L² is —(CH₂)_{q1}—C(O)—(NH)—NH—. In certain embodiments, L² is —(CH₂)_{q1}—C(O)(NH)—(CH₂)_{q3}—C(O)(NH)—(CH₂)_{q5}—. In certain embodiments, L² is —(CH₂)_{q1}—C(O)(NH)—(CH₂)_{q3}—C(O)—(NH)—NH—.

In certain embodiments, z1 is 1. In certain embodiments, z1 is 2.

In certain embodiments, the compound has a structure represented by formula IV-A or a pharmaceutically acceptable salt thereof:

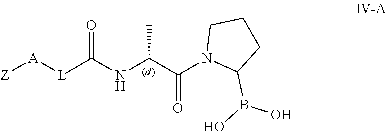

wherein
A is cycloalkyl, aryl, heteroaryl, or heterocyclyl;
Z is a radioactive halogen isotope or a trialkylammonium; and
L is a bond or a covalent linker.

In certain embodiments, the compound has a structure represented by formula IV-B or a pharmaceutically acceptable salt thereof:

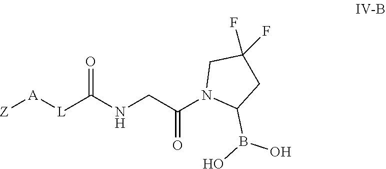

wherein
A is cycloalkyl, aryl, heteroaryl, or heterocyclyl;
Z is a radioactive halogen isotope or a trialkylammonium; and
L is a bond or a covalent linker (e.g., a substituted or unsubstituted C₁-C₁₂ alkylene, or a substituted or unsubstituted 2 to 12 membered heteroalkylene).

In certain embodiments, the compound has a structure represented by formula (IV-C) or a pharmaceutically acceptable salt thereof:

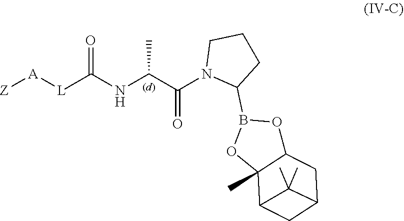

wherein:
A is cycloalkyl, aryl, heteroaryl, or heterocyclyl;
Z is a radioactive halogen isotope or a trialkylammonium; and
L is a bond, a substituted or unsubstituted C₁-C₁₂ alkylene, or a substituted or unsubstituted 2 to 12 membered heteroalkylene.

In certain embodiments, the compound has a structure represented by formula (IV-D) or a pharmaceutically acceptable salt thereof:

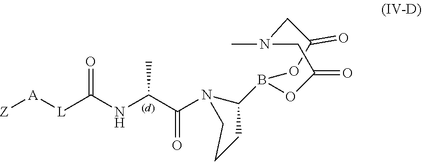

wherein:
A is cycloalkyl, aryl, heteroaryl, or heterocyclyl;
Z is a radioactive halogen isotope or a trialkylammonium; and
L is a bond, a substituted or unsubstituted C₁-C₁₂ alkylene, or a substituted or unsubstituted 2 to 12 membered heteroalkylene.

In certain embodiments, the compound has a structure represented by formula IV-E or a pharmaceutically acceptable salt thereof:

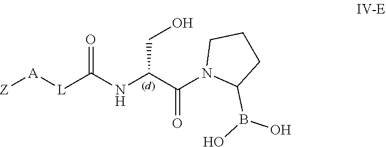

A is cycloalkyl, aryl, heteroaryl, or heterocyclyl;
Z is a radioactive halogen isotope or a trialkylammonium; and
L is a bond or a covalent linker.

In certain embodiments, the compound has a structure represented by formula IV-F or a pharmaceutically acceptable salt thereof:

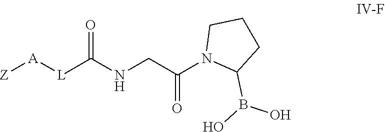

wherein
A is cycloalkyl, aryl, heteroaryl, or heterocyclyl;
Z is a radioactive halogen isotope or a trialkylammonium; and
L is a bond or a covalent linker.

In certain embodiments, the compound has a structure represented by formula (XIV-A), or a pharmaceutically acceptable salt thereof:

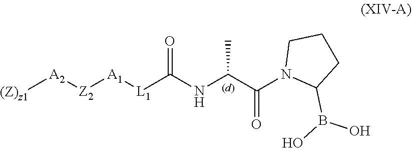

wherein:
each A₁, and A₂ is independently cycloalkyl, aryl, heteroaryl, or heterocyclyl;
Z is a radioactive halogen isotope or a trialkylammonium; and
each L₁, and L₂ is independently a bond, a substituted or unsubstituted C₁-C₁₂ alkylene, or a substituted or unsubstituted 2 to 12 membered heteroalkylene.

In certain embodiments, z1 is 1. In certain embodiments, z1 is 2.

In certain embodiments, the compound has a structure represented by formula (XXIV-A), or a pharmaceutically acceptable salt thereof:

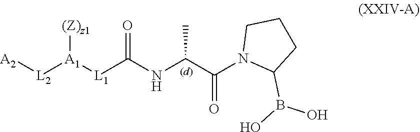

wherein:
each A₁, and A₂ is independently cycloalkyl, aryl, heteroaryl, or heterocyclyl;
Z is a radioactive halogen isotope or a trialkylammonium; and
each L₁, and L₂ is independently a bond, a substituted or unsubstituted C₁-C₁₂ alkylene, or a substituted or unsubstituted 2 to 12 membered heteroalkylene.

In certain embodiments, z1 is 1. In certain embodiments, z1 is 2.

In certain embodiments, the present disclosure provides compounds having a structure represented by formula (XIV-A) or a pharmaceutically acceptable salt thereof:

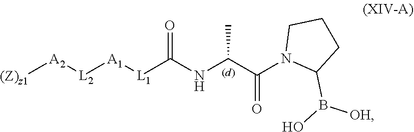

wherein:
A₁ is selected from the group consisting of

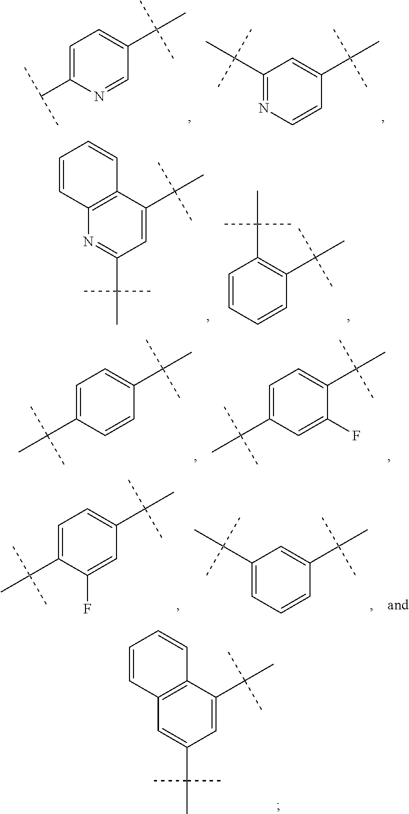

wherein [symbol] represents independently the connection to FAPx and to L₂; and
A₂ is selected from the group consisting of

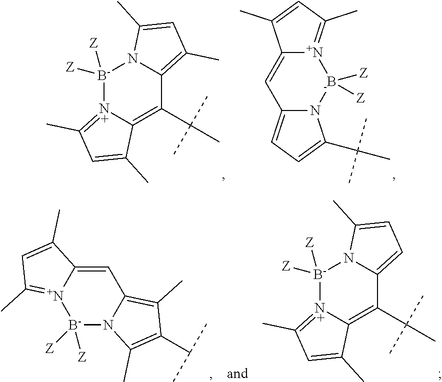

wherein [symbol] represents the connection to L₂; and
each Z is independently a radioactive halogen isotope or a trialkylammonium.

In certain embodiments, z1 is 1. In certain embodiments, z1 is 2.

In certain embodiments, L¹ is a bond. In certain embodiments, L¹ is a substituted or unsubstituted C₁-C₁₂ alkylene. In certain embodiments, L¹ is a substituted or unsubstituted 2 to 12 membered heteroalkylene.

In certain embodiments, L² is a substituted or unsubstituted C₁-C₁₂ alkylene. In certain embodiments, L² is a substituted or unsubstituted 2 to 12 membered heteroalkylene. In certain embodiments, L² is a oxo-substituted or unsubstituted 2 to 12 membered heteroalkylene. In certain embodiments, L² is —(CH₂)_{q1}—C(O)—(NH)_{q2}—(CH₂)_{q3}—; and each q1, q2, and q3 is independently 0 to 5. In certain embodiments, L² is —(CH₂)_{q1}—C(O)(NH)_{q2}—(CH₂)_{q3}—C(O)(NH)_{q4}—(CH₂)_{q5}—; and each q1, q2, q3, q4, and q5 is independently 0 to 5.

In certain embodiments, q1 is 1. In certain embodiments, q1 is 2. In certain embodiments, q1 is 3. In certain embodiments, q1 is 4. In certain embodiments, q1 is 5.

In certain embodiments, q2 is 0. In certain embodiments, q2 is 1. In certain embodiments, q2 is 2.

In certain embodiments, q3 is 0. In certain embodiments, q3 is 1. In certain embodiments, q3 is 2.

In certain embodiments, q4 is 0. In certain embodiments, q4 is 1. In certain embodiments, q4 is 2.

In certain embodiments, q5 is 0. In certain embodiments, q5 is 1. In certain embodiments, q5 is 2.

In certain embodiments, L² is —(CH₂)_{q1}—C(O)—(NH)—(CH₂)_{q3}—. In certain embodiments, L² is —(CH₂)_{q1}—C(O)—(NH)—NH—. In certain embodiments, L² is —(CH₂)_{q1}—C(O)(NH)—(CH₂)_{q3}—C(O)(NH)—(CH₂)_{q5}—. In certain embodiments, L² is —(CH₂)_{q1}—C(O)(NH)—(CH₂)_{q3}—C(O)—(NH)—NH—.

In certain embodiments, the present disclosure provides compounds having a structure represented by formula (XXIV-A) or a pharmaceutically acceptable salt thereof:

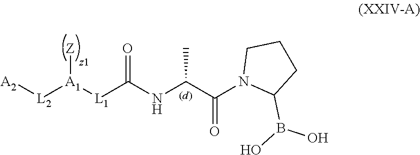

wherein
A₁ is selected from the group consisting

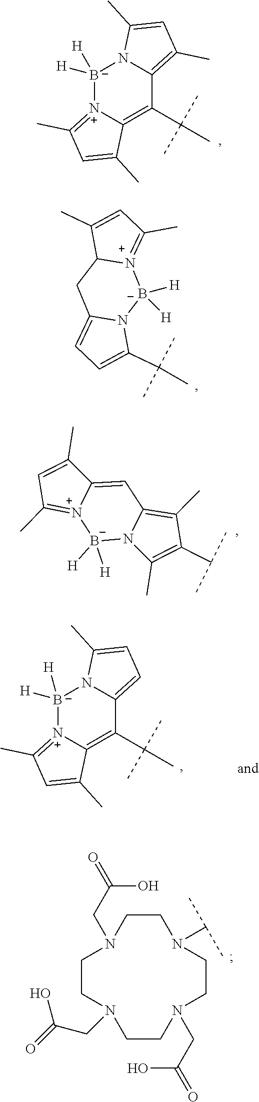

wherein [symbol] represents independently the connection to FAPx and to L₂;
A₂ is selected from the group consisting

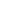

wherein [symbol] represents the connection to L₂; and
each Z is independently a radioactive halogen isotope or a trialkylammonium.

In certain embodiments, L¹ is a bond. In certain embodiments, L¹ is a substituted or unsubstituted C₁-C₁₂ alkylene. In certain embodiments, L¹ is a substituted or unsubstituted 2 to 12 membered heteroalkylene.

In certain embodiments, Vis a substituted or unsubstituted C₁-C₁₂ alkylene. In certain embodiments, L² is a substituted or unsubstituted 2 to 12 membered heteroalkylene. In certain embodiments, L² is a oxo-substituted or unsubstituted 2 to 12 membered heteroalkylene. In certain embodiments, L² is —(CH₂)_{q1}—C(O)—(NH)_{q2}—(CH₂)_{q3}— and each q1, q2, and q3 is independently 0 to 5. In certain embodiments, L² is —(CH₂)_{q1}—C(O)(NH)_{q2}—(CH₂)_{q3}—C(O)(NH)_{q4}—(CH₂)_{q5}— and each q1, q2, q3, q4, and q5 is independently 0 to 5.

In certain embodiments, q1 is 1. In certain embodiments, q1 is 2. In certain embodiments, q1 is 3. In certain embodiments, q1 is 4. In certain embodiments, q1 is 5.

In certain embodiments, q2 is 0. In certain embodiments, q2 is 1. In certain embodiments, q2 is 2.

In certain embodiments, q3 is 0. In certain embodiments, q3 is 1. In certain embodiments, q3 is 2.

In certain embodiments, q4 is 0. In certain embodiments, q4 is 1. In certain embodiments, q4 is 2.

In certain embodiments, q5 is 0. In certain embodiments, q5 is 1. In certain embodiments, q5 is 2.

In certain embodiments, L² is —(CH₂)_{q1}—C(O)—(NH)—(CH₂)_{q3}—. In certain embodiments, L² is (CH₂)_{q1}—C(O)—(NH)—NH—. In certain embodiments, L² is —(CH₂)_{q1}—C(O)(NH)—(CH₂)_{q3}—C(O)(NH)—(CH₂)_{q5}—. In certain embodiments, L² is —(CH₂)_{q1}—C(O)(NH)—(CH₂)_{q3}—C(O)—(NH)—NH—.

In certain embodiments, the compound has a structure represented by formula (XIV-B), or a pharmaceutically acceptable salt thereof:

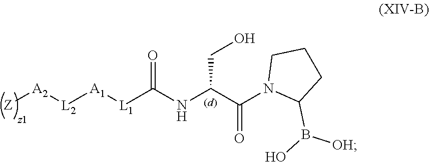

wherein A₁, A₂, Z, L₁, L₂ and z1 are as described above.

In certain embodiments, the compound has a structure represented by formula (XXIV-B), or a pharmaceutically acceptable salt thereof:

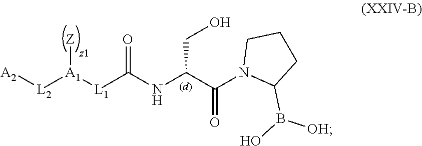

wherein A₁, A₂, Z, L₁, L₂ and z1 are as described above.

In certain embodiments, the compound has a structure represented by formula (XIV-C), or a pharmaceutically acceptable salt thereof:

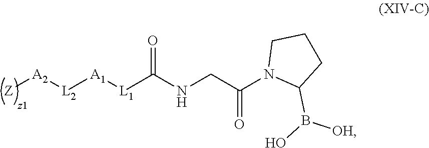

wherein A₁, A₂, Z, L₁, L₂ and z1 are as described above.

In certain embodiments, the compound has a structure represented by formula (XXIV-C), or a pharmaceutically acceptable salt thereof:

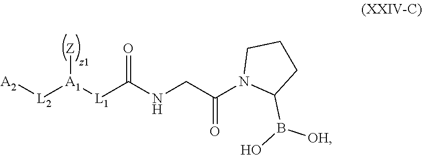

wherein A₁, A₂, Z, L₁, L₂ and z1 are as described above.

In certain embodiments, the compound has a structure represented by formula V or a pharmaceutically acceptable salt thereof:

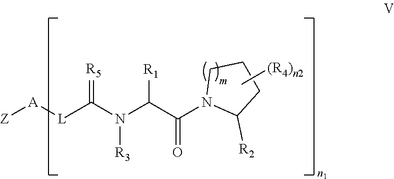

wherein,
A is cycloalkyl, aryl, heteroaryl, or heterocyclyl;
Z is a radioactive halogen isotope or a trialkylammonium;
L is a bond or a covalent linker (e.g., a substituted or unsubstituted C₁-C₁₂ alkylene, or a substituted or unsubstituted 2 to 12 membered heteroalkylene);
R₁ is H or alkyl;
R₂ is a moiety which covalently binds to a side chain of an amino acid in an active site of FAP;
R₃ is H or alkyl;
R₄ is alkyl, hydroxyl, amino, or halo;
R₅ is O or S;
m is 1-3;
n₁ is 2-6; and
n₂ is 0-7.

In certain embodiments, the compound has a structure represented by formula VI or a pharmaceutically acceptable salt thereof:

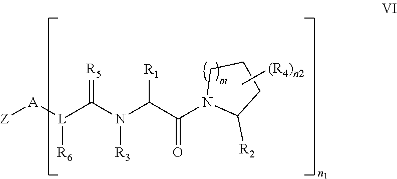

wherein,
A is cycloalkyl, aryl, heteroaryl, or heterocyclyl;
Z is a radioactive halogen isotope or a trialkylammonium;
L is a bond or a covalent linker (e.g., a substituted or unsubstituted C₁-C₂ alkylene, or a substituted or unsubstituted 2 to 12 membered heteroalkylene);
R₁ is H or alkyl;
R₂ is a moiety which covalently binds to a side chain of an amino acid in an active site of FAP;
R₃ is H or alkyl;
R₄ is alkyl, hydroxyl, amino, or halo;
R₅ is O or S;
R_{b} is a moiety which modifies the pharmacokinetics or bio-distribution of the compound;
m is 1-3;
n₁ is 2-6; and
n₂ is 0-7.

In certain embodiments, the compound has a structure represented by formula VII or a pharmaceutically acceptable salt thereof:

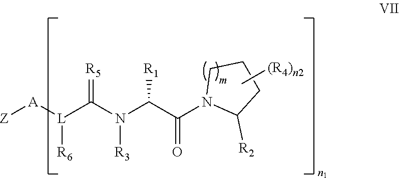

wherein,
A is cycloalkyl, aryl, heteroaryl, or heterocyclyl;
Z is a radioactive halogen isotope or a trialkylammonium;
L is a bond or a covalent linker (e.g., a substituted or unsubstituted C₁-C₁₂ alkylene, or a substituted or unsubstituted 2 to 12 membered heteroalkylene);
R₁ is H or alkyl;
R₂ is a moiety which covalently binds to a side chain of an amino acid in an active site of FAP;
R₃ is H or alkyl;
R₄ is alkyl, hydroxyl, amino, or halo;
R₅ is O or S;
R₆ is a moiety which modifies the pharmacokinetics or bio-distribution of the compound;
m is 1-3;
n₁ is 2-6; and
n₂ is 0-7.

In certain embodiments, R₁ is H. In certain embodiments, R₁ is H, or (C₁-C₆)alkyl (e.g., methyl or ethyl), which is optionally substituted with —OH. In certain embodiments, R¹ is —CH₃, or —CH₂—OH.

In certain embodiments, R₂ is B(Y¹)(Y²), CN, or formyl; wherein Y¹ and Y² are each hydroxyl; or Y¹ and Y² together with the boron atom to which they are attached combine to form a moiety which is hydrolysable to a boronic acid. In certain embodiments, Y¹ and Y² together with the boron atom to which they are attached combine to form a 5- to 8-membered ring. In certain embodiments, R₂ is B(OH)₂.

In certain embodiments, R₃ is (C₁-C₆)alkyl. In certain embodiments, R₃ is H.

In certain embodiments, R₄ is (C₁-C₆)alkyl. In certain embodiments, R₄ is halo (e.g., fluorine).

In certain embodiments, R₅ is O.

In certain embodiments, R₆ is a moiety which modifies the serum half-life of the compound or the tumor distribution of the compound. In certain embodiment, R₆ is a non-proteinaceous half-life extending moiety (e.g., a water soluble polymer such as polyethylene glycol (PEG) or discrete PEG, hydroxyethyl starch (HES)), a lipid, a branched or unbranched acyl group, a branched or unbranched C8-C30 acyl group, a branched or unbranched alkyl group, and a branched or unbranched C8-C30 alkyl group). In certain embodiments, R₆ is a proteinaceous half-life extending moiety (e.g., serum albumin, transferrin, an adnectin (e.g., albumin-binding or pharmacokinetics extending (PKE) adnectins), Fc domain unstructured polypeptide (e.g., XTEN polypeptide, PAS polypeptide, conformationally disordered polypeptide sequences composed of the amino acids Pro, Ala, and/or Ser, or a fragment of any of the foregoing).

In certain embodiments, n₂ is 2. In certain embodiments, is 0.

In certain embodiments, Z is ¹⁸F or ¹⁹F. In certain embodiments, Z is ¹³¹I, ¹²³, ¹²⁴I, or ¹²⁵I. In certain embodiments, Z is ⁷⁶Br or ⁷⁵Br. In certain embodiments, Z is trialkylammonium.

In certain embodiments, Z is N(R₇)₃; and each R₇ is (C₁-C₆)alkyl (e.g., methyl or ethyl).

In certain embodiments, L is a bond.

In certain embodiments, A is a monocyclic cycloalkyl, monocyclic aryl, monocyclic heteroaryl, or monocyclic heterocyclyl. In certain embodiments, A is a bicyclic cycloalkyl, monocyclic aryl, bicyclic heteroaryl, or bicyclic heterocyclyl. In certain embodiments, the bicycle is fused, bridged, or spirocyclic. In certain embodiment, A is a polycyclic cycloalkyl, polycyclic aryl, polycyclic heteroaryl, or polycyclic heterocyclyl. In certain embodiments, A is heteroaryl. In certain embodiments, comprises 4-12 ring atoms. In certain embodiments, comprises 5-12 ring atoms. In certain embodiments, comprises 6-12 ring atoms. In certain embodiments, A is aryl. In certain embodiments, A further comprises 1-4 hetero ring atoms selected from the group consisting of N, O, and S. In certain embodiments, A is selected from the group consisting of pentalene, indene, naphthalene, azulene, hetalene, biphenylene, indacene, acenaphthylene, fluorene, phenalene, phenanthrene, anthracene, fluoranthene, acephenanthrylene, aceanthrylene, triphenylene, pyrne, chrysene, naphthacene, pleiadene, picene and perylene. In certain embodiments, A is selected from the group consisting of azepine, benzofuran, benzopyran, benzothine, chromene, cinnoline, diazepine, diazepinepyrrolopyridine, dioxin, furan, furazan, imidazole, imidazothiazole, indazole, indole, isobenzofuran, isoindole, isopyrazole, isoquinoline, isothianaphthelene, isothiazole, naphthyridine, oxadiazole, oxatriazole, oxazole, oxepin, phthalazine, pteridine, purine, pyran, pyrazine ring, pyrazole, pyridazine, pyridine, pyrimidine, pyrrole, quinazoline, quinolizine, quinoxaline, tetrazole, thaidiazole, thianapthalene, thiazole, thienopyrrole, thiepin, thiophene, ring, and triazole. In certain embodiments, A is selected from the group consisting of pyridine, quinolone, and isoindole. In certain embodiments, A is selected from the group consisting of phenyl and naphthalene.

In certain embodiments, A is selected from the group consisting of:

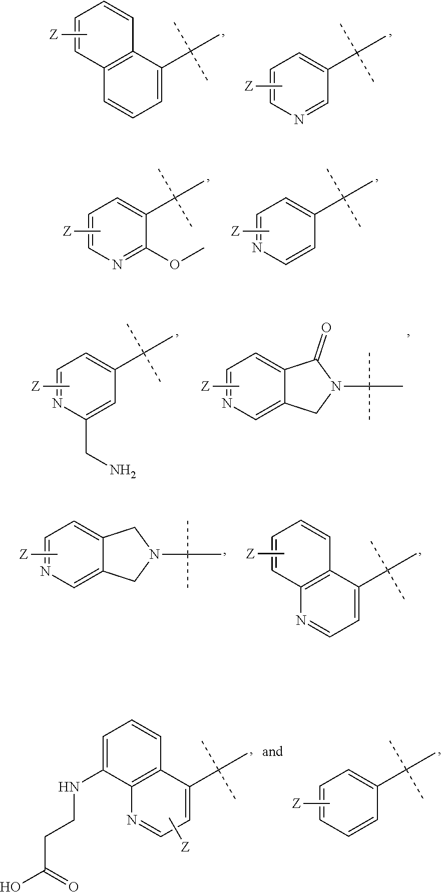

or a pharmaceutically acceptable salt thereof;
Z is a radioactive halogen isotope or a trialkylammonium; and
[symbol] represents the connection to FAPx.

In certain embodiments, A is selected from the group consisting of:

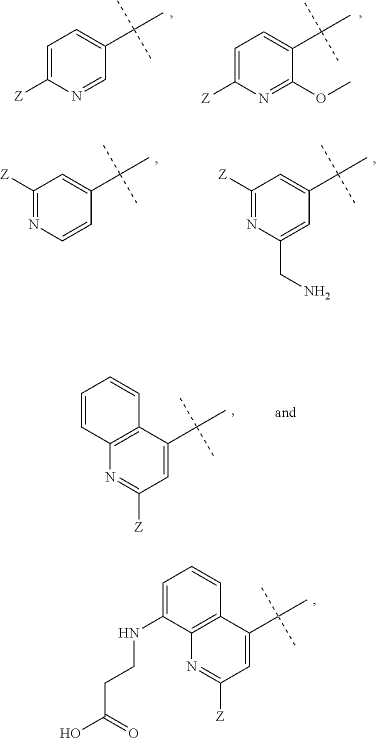

or a pharmaceutically acceptable salt thereof;
Z is a radioactive halogen isotope or a trialkylammonium; and
[symbol] represents the connection to FAPx.

In certain embodiments, A is selected from the group consisting of:

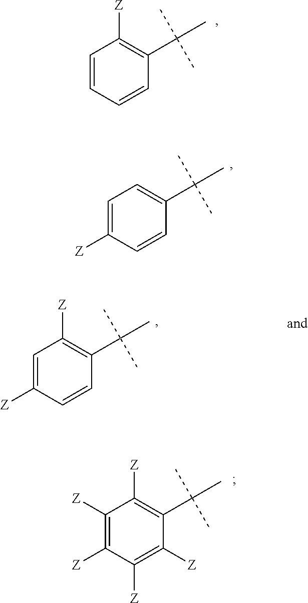

Z is a radioactive halogen isotope or a trialkylammonium; and
[symbol] represents the connection to FAPx.

In certain embodiments, A is selected from the group consisting of:

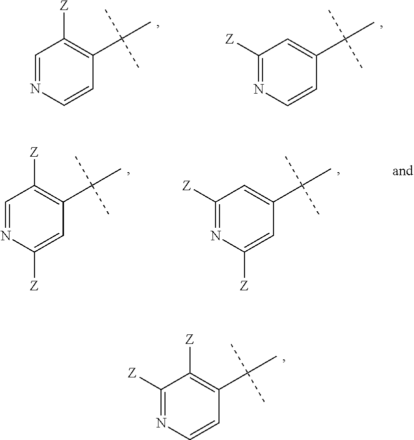

or a pharmaceutically acceptable salt thereof;
Z is a radioactive halogen isotope or a trialkylammonium; and
[symbol] represents the connection to FAPx.

In certain embodiments, the compound of Formula (I-A) is selected from the group consisting of:

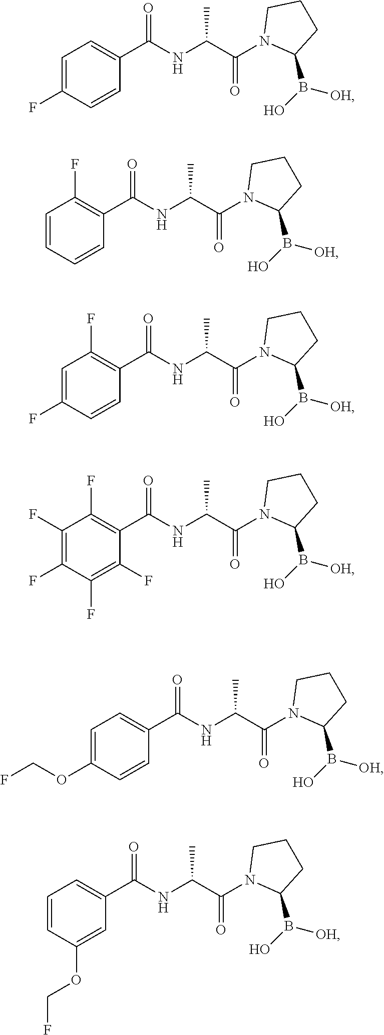 [SMILES: C[C@@H](NC(=O)c1ccc(F)cc1)C(=O)N1CCC[C@H]1B(O)O ; C[C@@H](NC(=O)c1ccccc1F)C(=O)N1CCC[C@H]1B(O)O ; C[C@@H](NC(=O)c1ccc(F)cc1F)C(=O)N1CCC[C@H]1B(O)O ; C[C@@H](NC(=O)c1c(F)c(F)c(F)c(F)c1F)C(=O)N1CCC[C@H]1B(O)O ; C[C@@H](NC(=O)c1ccc(OCF)cc1)C(=O)N1CCC[C@H]1B(O)O ; C[C@@H](NC(=O)c1cccc(OCF)c1)C(=O)N1CCC[C@H]1B(O)O]

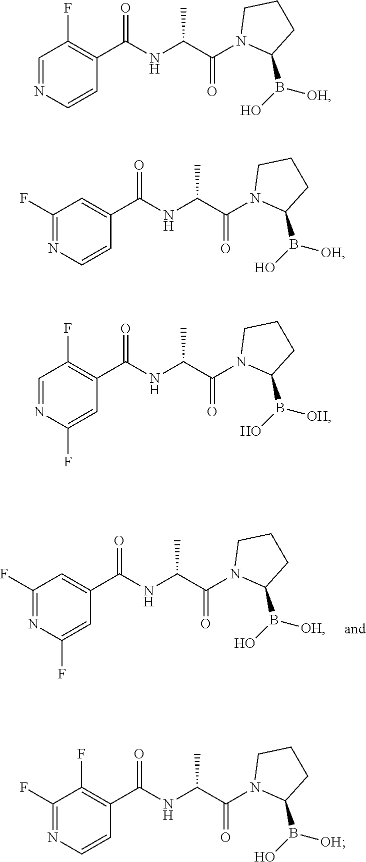 [SMILES: C[C@@H](NC(=O)c1ccncc1F)C(=O)N1CCC[C@H]1B(O)O ; C[C@@H](NC(=O)c1ccnc(F)c1)C(=O)N1CCC[C@H]1B(O)O ; C[C@@H](NC(=O)c1cc(F)ncc1F)C(=O)N1CCC[C@H]1B(O)O ; C[C@@H](NC(=O)c1cc(F)nc(F)c1)C(=O)N1CCC[C@H]1B(O)O ; C[C@@H](NC(=O)c1ccnc(F)c1F)C(=O)N1CCC[C@H]1B(O)O]

or a pharmaceutically acceptable salt thereof.

In certain embodiments, at least one instance of F is enriched in ¹⁸F compared to the natural abundance.

In certain embodiments, A is selected from the group consisting of:

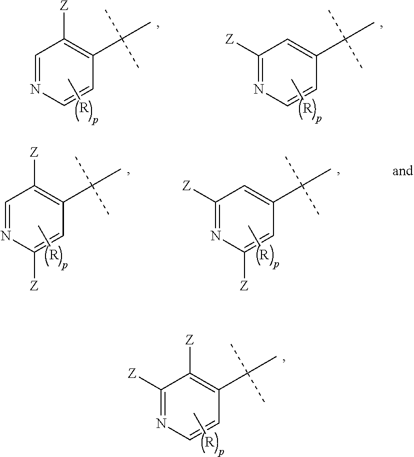

or a pharmaceutically acceptable salt thereof;
Z is a radioactive halogen isotope or a trialkylammonium; p is 1 to 3, and
[symbol] represents the connection to FAPx.

In certain embodiments, the compound of Formula (I-A) is selected from the group consisting of:

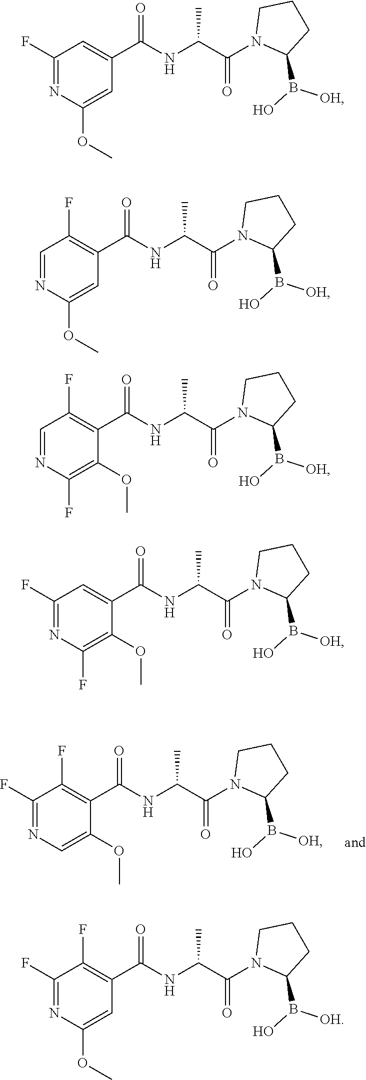

In certain embodiments, A is selected from the group consisting of:

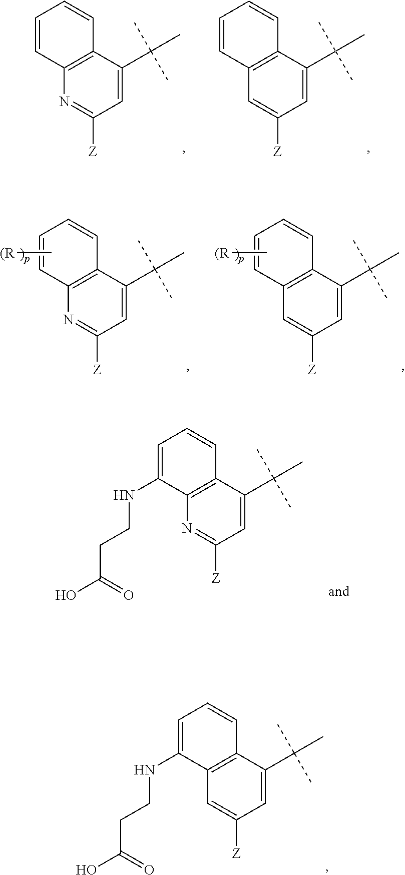

or a pharmaceutically acceptable salt thereof;
Z is a radioactive halogen isotope or a trialkylammonium; and
[symbol] represents the connection to FAPx.

In certain embodiments, at least one instance of F is enriched in ¹⁸F compared to the natural abundance.

In certain embodiments, the compound of Formula (I-A) is selected from the group consisting of:

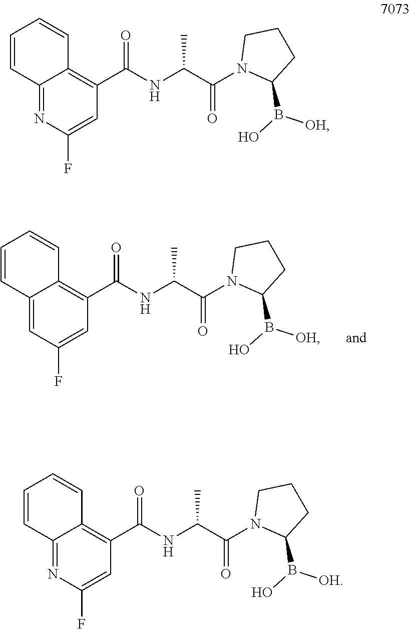 [SMILES: C[C@@H](NC(=O)c1cc(F)nc2ccccc12)C(=O)N1CCC[C@H]1B(O)O ; C[C@@H](NC(=O)c1cc(F)cc2ccccc12)C(=O)N1CCC[C@H]1B(O)O ; C[C@@H](NC(=O)c1cc(F)nc2ccccc12)C(=O)N1CCC[C@H]1B(O)O]

In certain embodiments, at least one instance of F is enriched in ¹⁸F compared to the natural abundance.

In certain embodiments the compounds of Formula IV-C and (IV-D include:

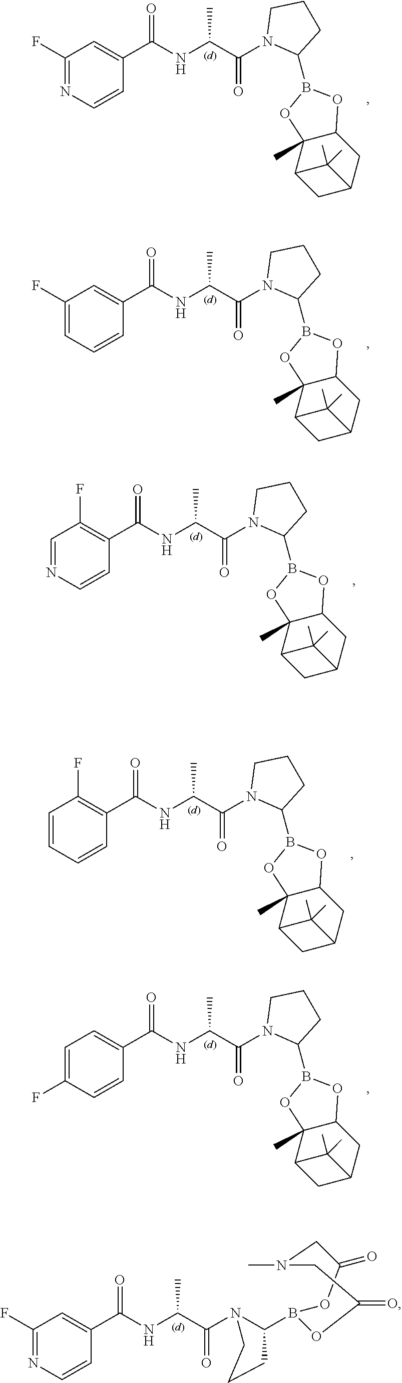

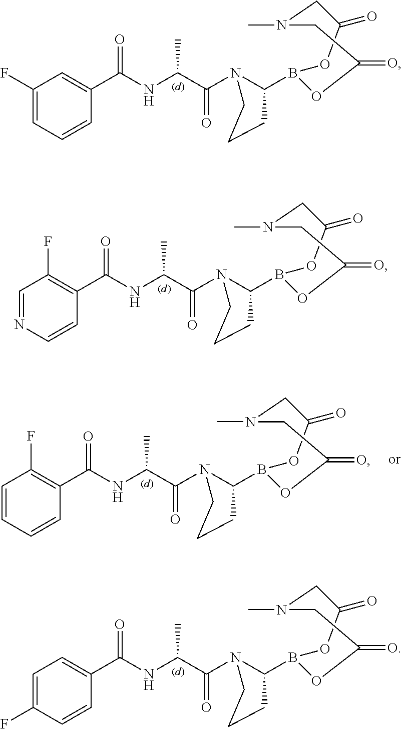

In certain embodiments, the compound of Formula (XI) is selected from the group consisting of:

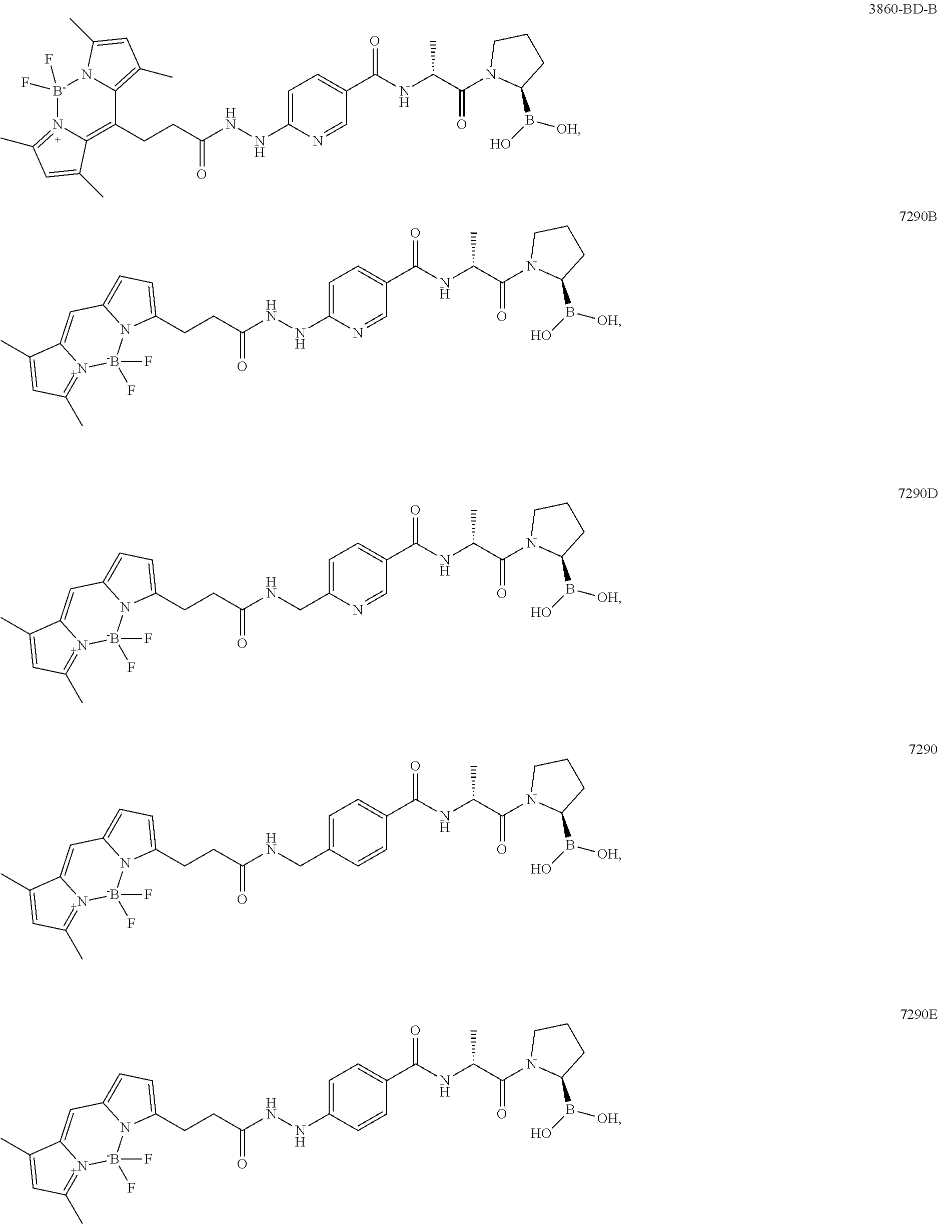

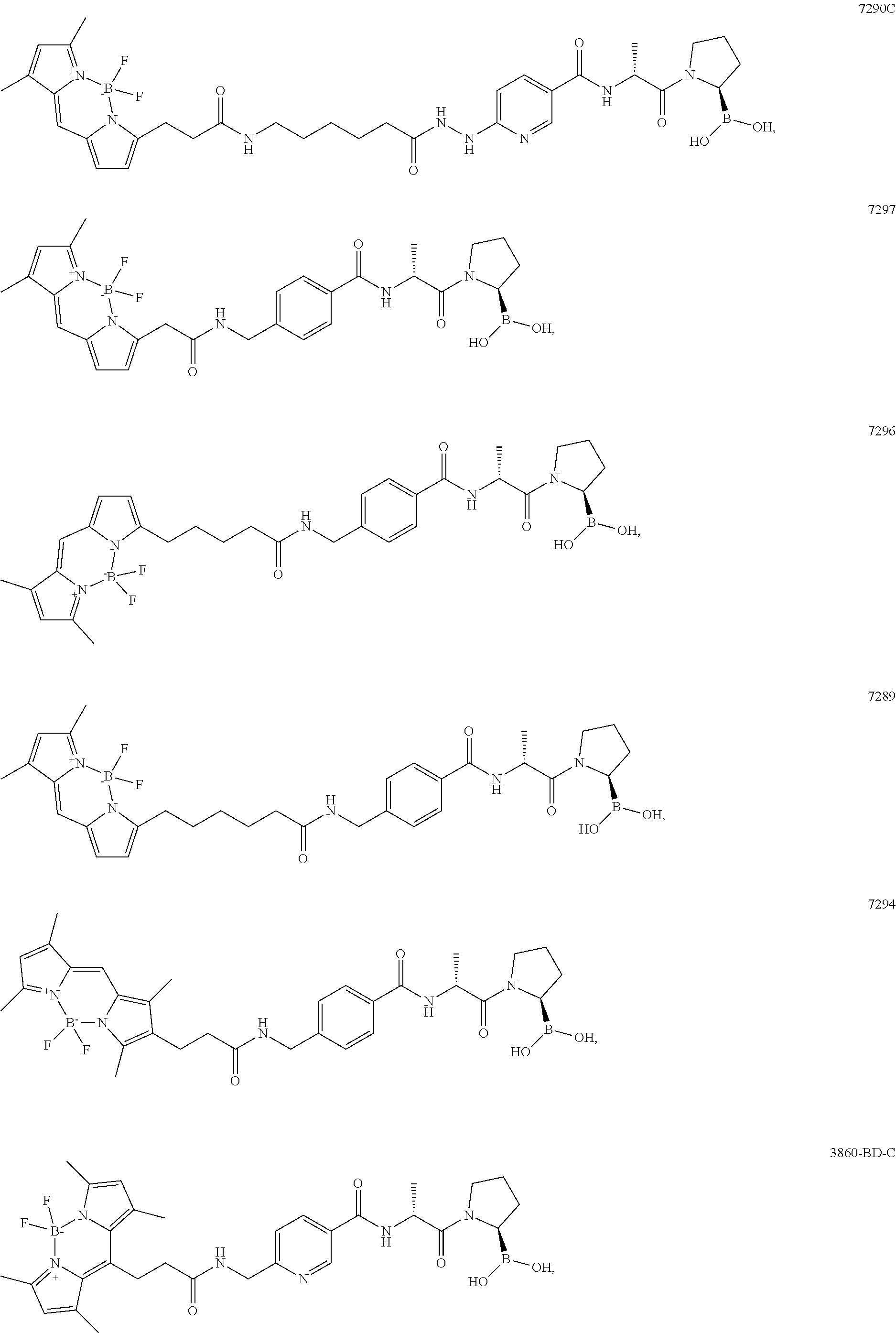

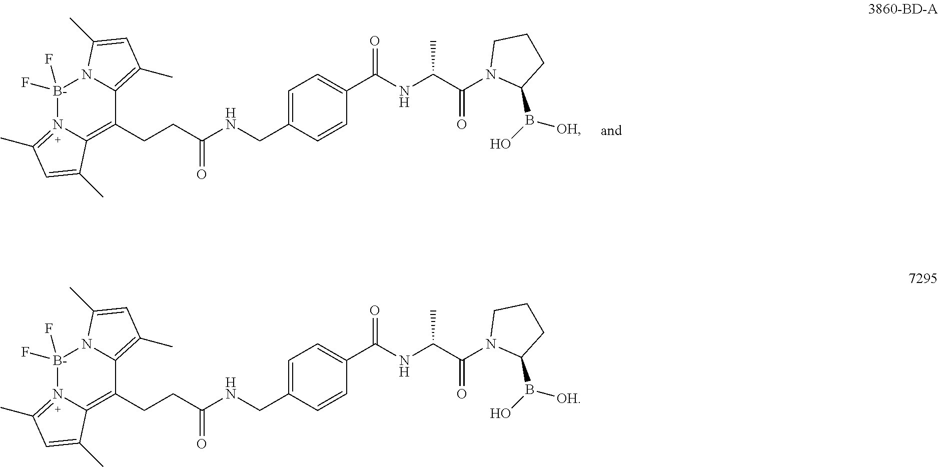

In certain embodiments, at least one instance of F is enriched in ¹⁸F compared to the natural abundance.

In certain embodiments, the compound of Formula (XXI) is selected from the group consisting of:

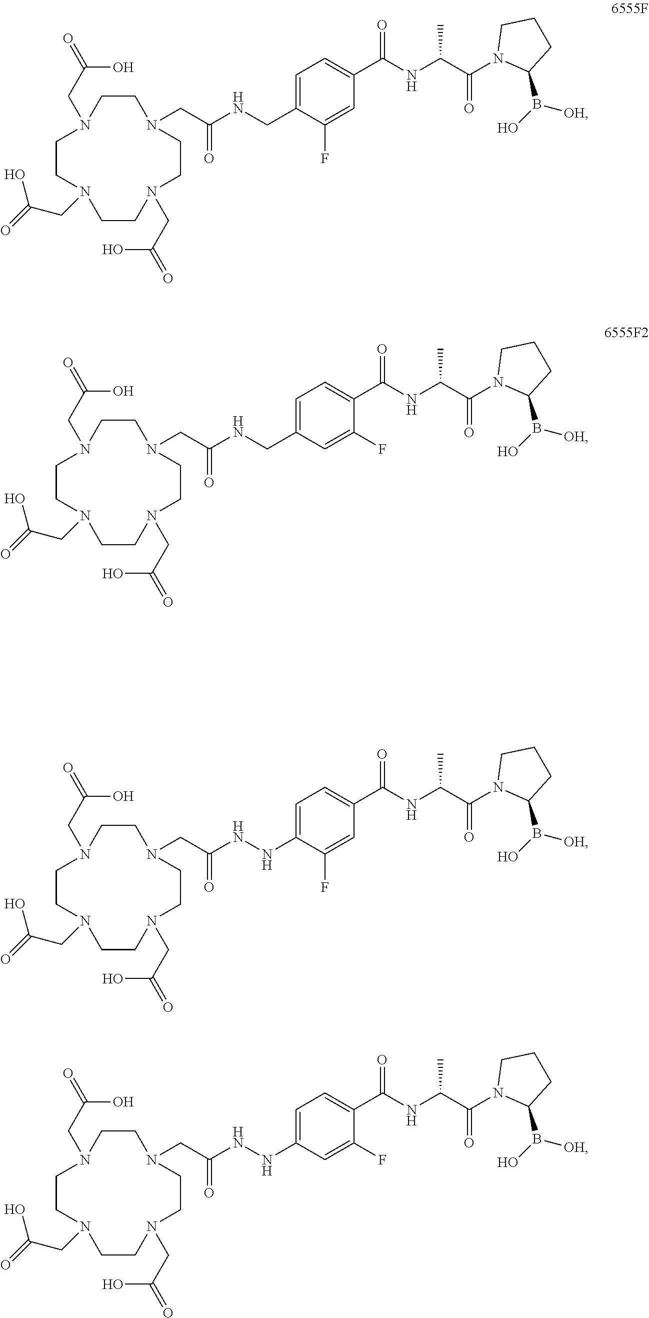 [SMILES: C[C@@H](NC(=O)c1ccc(CNC(=O)CN2CCN(CC(=O)O)CCN(CC(=O)O)CCN(CC(=O)O)CC2)c(F)c1)C(=O)N1CCC[C@H]1B(O)O ; C[C@@H](NC(=O)c1ccc(CNC(=O)CN2CCN(CC(=O)O)CCN(CC(=O)O)CCN(CC(=O)O)CC2)cc1F)C(=O)N1CCC[C@H]1B(O)O ; C[C@@H](NC(=O)c1ccc(NNC(=O)CN2CCN(CC(=O)O)CCN(CC(=O)O)CCN(CC(=O)O)CC2)c(F)c1)C(=O)N1CCC[C@H]1B(O)O ; C[C@@H](NC(=O)c1ccc(NNC(=O)CN2CCN(CC(=O)O)CCN(CC(=O)O)CCN(CC(=O)O)CC2)cc1F)C(=O)N1CCC[C@H]1B(O)O]

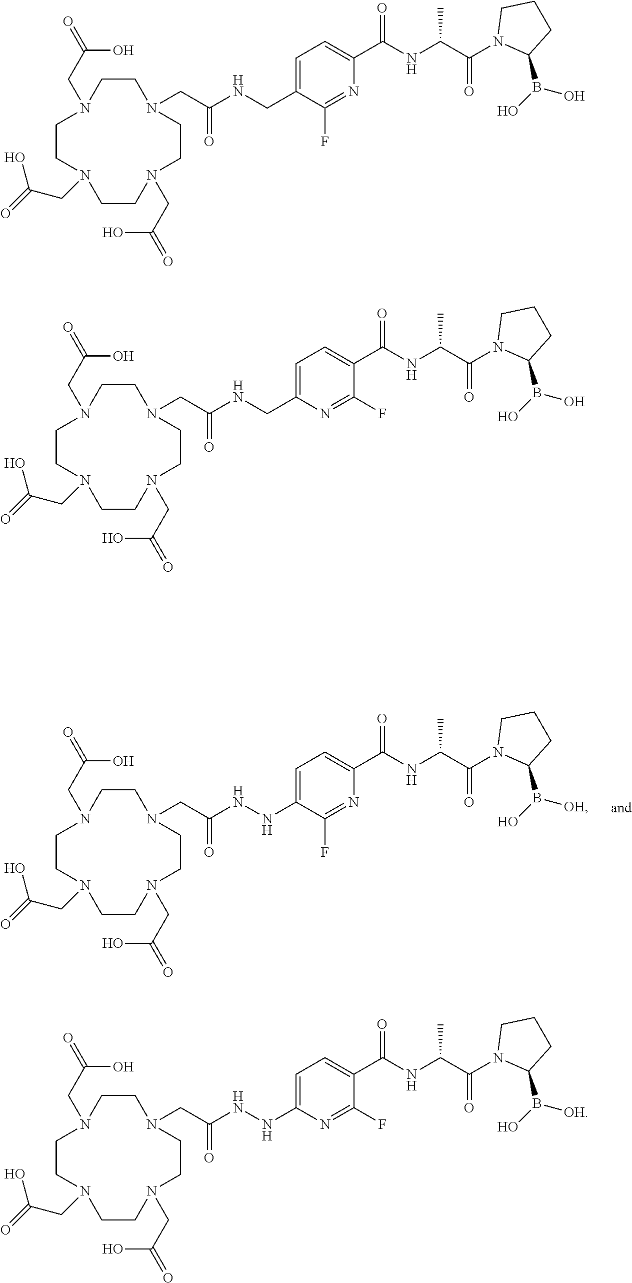 [SMILES: C[C@@H](NC(=O)c1ccc(CNC(=O)CN2CCN(CC(=O)O)CCN(CC(=O)O)CCN(CC(=O)O)CC2)c(F)n1)C(=O)N1CCC[C@H]1B(O)O ; C[C@@H](NC(=O)c1ccc(CNC(=O)CN2CCN(CC(=O)O)CCN(CC(=O)O)CCN(CC(=O)O)CC2)nc1F)C(=O)N1CCC[C@H]1B(O)O ; C[C@@H](NC(=O)c1ccc(NNC(=O)CN2CCN(CC(=O)O)CCN(CC(=O)O)CCN(CC(=O)O)CC2)c(F)n1)C(=O)N1CCC[C@H]1B(O)O ; C[C@@H](NC(=O)c1ccc(NNC(=O)CN2CCN(CC(=O)O)CCN(CC(=O)O)CCN(CC(=O)O)CC2)nc1F)C(=O)N1CCC[C@H]1B(O)O]

In certain embodiments, at least one instance of F is enriched in ¹⁸F compared to the natural abundance.

In another aspect, the present disclosure provides pharmaceutical compositions comprising a compound disclosed herein; and a pharmaceutically acceptable excipient.

In another aspect, the present disclosure provides methods of method of detecting a cell in a subject, comprising the steps of:

administering to the subject a therapeutically effective amount of a compound disclosed
herein or a pharmaceutically acceptable salt thereof; and obtaining an image of the subject.

In certain embodiments, the cell overexpresses FAP. In certain embodiments, the cell is a cancer cell (e.g., a prostate, pancreatic, colon, or breast cancer cell).

In certain embodiments, the image is obtained using a positron emission tomography scanner.

In another aspect, the present disclosure provides methods of treating cancer in a subject, comprising administering to the subject a therapeutically effective amount of a compound disclosed herein or a pharmaceutically acceptable salt thereof. In certain embodiments, the cancer is prostate cancer, pancreatic cancer, colon cancer, or breast cancer.

In another aspect, the present disclosure provides methods of making compounds disclosed herein, wherein Z is a radioactive halogen isotope and A is aryl or heteroaryl, comprising contacting a compound disclosed herein wherein Z is trialkylammonium and A is aryl or heteroaryl with the radioactive halogen isotope, thereby making the compound wherein Z is a radioactive halogen isotope and A is aryl or heteroaryl.

In certain embodiments, the radioactive halogen isotope is Z is ¹⁸F or ¹⁹F. In certain embodiments, the radioactive halogen isotope is ¹³¹I, ¹²³, ¹²⁴I, or ¹²⁵I. In certain embodiments, the radioactive halogen isotope is ⁷⁶Br or ⁷⁵Br.

In certain embodiments, the method is performed at about 30° C. to about 70° C. In certain embodiments, the method is performed at about 50° C.

In certain embodiments, the method is performed for about 20 seconds to about 10 minutes. In certain embodiments, the method is performed for about 150 seconds. In certain embodiments, the method is performed for about 200 seconds. In certain embodiments, the method is performed for about 6 minutes.

In certain embodiments, the method is performed in a microwave.

In certain embodiments, the power of the microwave radiation is about 40 to about 60 watts. In certain embodiments, the power of the microwave radiation is about 50 watts.

In another aspect, the present disclosure provides kits comprising a compound disclosed herein wherein Z is trialkylammonium and instructions for making the compounds disclosed herein wherein Z is a radioactive halogen isotope.

In another aspect, the present disclosure provides kits comprising a compound disclosed herein wherein Z is a radioactive halogen isotope and instructions for performing the methods disclosed herein relating to the diagnosis and/or treatment of cancer.

### Pharmaceutical Compositions

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs.

The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a selfemulsifying drug delivery system or a selfmicroemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the invention may be used alone or conjointly administered with another type of therapeutic agent.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known. The ability of such agents to inhibit AR or promote AR degradation may render them suitable as “therapeutic agents” in the methods and compositions of this disclosure.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(OXO-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆ alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS-.

The term “amide”, as used herein, refers to a group

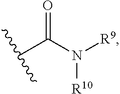

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

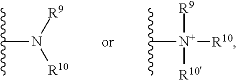

wherein R⁹, R¹⁰, and R^{10′} each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

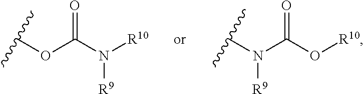

wherein R⁹ and R¹⁰ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “cycloalkyl” includes substituted or unsubstituted non-aromatic single ring structures, preferably 4- to 8-membered rings, more preferably 4- to 6-membered rings. The term “cycloalkyl” also includes polycyclic ring systems having two or more cyclic rings in which two or more carbons are common to two adjoining rings wherein at least one of the rings is cycloalkyl and the substituent (e.g., R¹⁰⁰) is attached to the cycloalkyl ring, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, pyrimidine, denzodioxane, tetrahydroquinoline, and the like.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

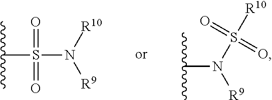

wherein R⁹ and R¹⁰ independently represents hydrogen or hydrocarbyl.

The term “sulfoxide” is art-recognized and refers to the group-S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁹ or —SC(O)R⁹
wherein R⁹ represents a hydrocarbyl.

The term “thioether”, as used herein, is equivalent to an ether, wherein the oxygen is replaced with a sulfur.

The term “urea” is art-recognized and may be represented by the general formula

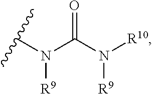

wherein R⁹ and R¹⁰ independently represent hydrogen or a hydrocarbyl.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Formula I. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula I or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the compounds useful in the methods and compositions of this disclosure have at least one stereogenic center in their structure. This stereogenic center may be present in a R or a δ configuration, said R and δ notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form the compound of the present disclosure (e.g., compounds of formula I). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of Formula I. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “Log of solubility”, “LogS” or “logS” as used herein is used in the art to quantify the aqueous solubility of a compound. The aqueous solubility of a compound significantly affects its absorption and distribution characteristics. A low solubility often goes along with a poor absorption. LogS value is a unit stripped logarithm (base 10) of the solubility measured in mol/liter.

### Kits and Methods

As discussed above, kits are provided comprising, consisting essentially of or consisting of at least one compound including a compound of any of the formulae disclosed herein (including formulae I, XI, XXI, IL XII, XXII, III, XIII, XXIII, IV-A, IV-B, IV-C, IV-D, XIV-A, XXIV-A and/or XIV-A), and instructions for the diagnosis or treatment of a disease.

And still another aspect of the invention provides methods for diagnosing, imaging or reducing tissue overexpressing FAP in an animal (preferably a human patient), comprising administering to the animal a compound of any of the formulae disclosed herein (including formulae I, XI, XXI, II, XII, XXII, III, XIII, XXIII, IV-A, IV-B, IV-C, IV-D, XIV-A, XXIV-A and/or XIV-A). In some embodiments, the tissue overexpressing FAP is a tumor, especially a solid tumor. In some embodiments, the tumor is a tumor selected from the group consisting of: colorectal tumor, pancreatic tumor, lung tumor, ovarian tumor, liver tumor, breast tumor, kidney tumor, prostate tumor, neuroendocrine tumor, gastrointestinal tumor, melanoma, cervical tumor, bladder tumor, glioblastoma, and head and neck tumor. In some embodiments, the tumor is a colorectal tumor. In some embodiments, the tumor is an ovarian tumor. In some embodiments, the tumor is a lung tumor. In some embodiments, the tumor is a pancreatic tumor. In some embodiments, the tumor is a melanoma tumor. In some embodiments, the tumor is a bladder tumor. In some embodiments, the tumor is a prostate tumor.

To further illustrate, the subject a compound of any one of the formulae disclosed herein (including formulae I, XI, XXI, II, XII, XXII, III, XIII, XXIII, IV-A, IV-B, IV-C, IV-D, XIV-A, XXIV-A and/or XIV-A can be used to treat patients suffering from cancer, such as osteosarcoma, rhabdomyosarcoma, neuroblastoma, kidney cancer, leukemia, renal transitional cell cancer, bladder cancer, Wilm's cancer, ovarian cancer, pancreatic cancer, breast cancer (including triple negative breast cancer), prostate cancer, bone cancer, lung cancer (e.g., small cell or non-small cell lung cancer), gastric cancer, colorectal cancer, cervical cancer, synovial sarcoma, head and neck cancer, squamous cell carcinoma, multiple myeloma, renal cell cancer, retinoblastoma, hepatoblastoma, hepatocellular carcinoma, melanoma, rhabdoid tumor of the kidney, Ewing's sarcoma, chondrosarcoma, brain cancer, glioblastoma, meningioma, pituitary adenoma, vestibular schwannoma, a primitive neuroectodermal tumor, medulloblastoma, astrocytoma, anaplastic astrocytoma, oligodendroglioma, ependymoma, choroid plexus papilloma, polycythemia vera, thrombocythemia, idiopathic myelfibrosis, soft tissue sarcoma, thyroid cancer, endometrial cancer, carcinoid cancer or liver cancer, breast cancer or gastric cancer.

In an additional aspect, the cancer is metastatic cancer, e.g., of the varieties described above. In some embodiments, in addition to administering an FAP-targeted agent described herein, the method or treatment further comprises administering at least one additional immune response stimulating agent. In some embodiments, the additional immune response stimulating agent includes, but is not limited to, a colony stimulating factor (e.g., granulocyte-macrophage colony stimulating factor (GM-CSF), macrophage colony stimulating factor (M-CSF), granulocyte colony stimulating factor (G-CSF), stem cell factor (SCF)), an interleukin (e.g., IL-1, IL2, IL-3, IL-7, IL-12, IL-15, IL-18), a checkpoint inhibitor, an antibody that blocks immunosuppressive functions (e.g., an anti-CTLA-4 antibody, anti-CD28 antibody, anti-CD3 antibody), a toll-like receptor (e.g., TLR4, TLR7, TLR9), or a member of the B7 family (e.g., CD80, CD86). An additional immune response stimulating agent can be administered prior to, concurrently with, and/or subsequently to, administration of the FAP-targeted agent.

Pharmaceutical compositions comprising a compound of any one of the formulae disclosed herein (including formulae I, XI, XXI, II, XII, XXII, III, XIII, XXIII, IV-A, IV-B, IV-C, IV-D, XIV-A, XXIV-A and/or XIV-A) and an immune response stimulating agent(s) are also provided. In some embodiments, the immune response stimulating agent comprises 1, 2, 3, or more immune response stimulating agents.

In some embodiments, in addition to administering a compound of any one of the formulae disclosed herein (including formulae I, XI, XXI, II, XII, XXII, III, XIII, XXIII, IV-A, IV-B, IV-C, IV-D, XIV-A, XXIV-A and/or XIV-A), the method or treatment further comprises administering at least one additional therapeutic agent. An additional therapeutic agent can be administered prior to, concurrently with, and/or subsequently to, administration of a compound of any of the formulae disclosed herein (including formulae I, XI, XXI, IL XII, XXII, III, XIII, XXIII, IV-A, IV-B, IV-C, IV-D, XIV-A, XXIV-A and/or XIV-A). Pharmaceutical compositions comprising a compound of any of the formulae disclosed herein (including formulae I, XI, XXI, H, XII, XXII, III, XIII, XXIII, IV-A, IV-B, IV-C, IV-D, XIV-A, XXIV-A and/or XIV-A) and the additional therapeutic agent(s) are also provided. In some embodiments, the at least one additional therapeutic agent comprises 1, 2, 3, or more additional therapeutic agents. Combination therapy with two or more therapeutic agents often uses agents that work by different mechanisms of action, although this is not required. Combination therapy using agents with different mechanisms of action may result in additive or synergetic effects. Combination therapy may allow for a lower dose of each agent than is used in monotherapy, thereby reducing toxic side effects and/or increasing the therapeutic index of a compound of any of the formulae disclosed herein (including formulae I, XI, XXI, II, XII, XXII, III, XIII, XXIII, IV-A, IV-B, IV-C, IV-D, XIV-A, XXIV-A and/or XIV-A).

### EXAMPLES

The invention now being generally described, it will be more readily understood by reference to the following examples which are included merely for purposes of illustration of certain aspects and embodiments of the present invention, and are not intended to limit the invention.

### Example 1: Preparation of Exemplary Compounds of the Disclosure

### 1. Synthetic Method Suitable for the Preparation of [¹⁸F]-Labelled N-(2-Fuoropyrid-ine-4-Carbonyl)-D-Ala-boroPro (3099D)

### (1) Method A.

The Method A via a three steps sequence is summarized in Scheme 1. Firstly, the 2-N, N, N-trimethylammonium pyridine carboxylate tert-butyl ester triflate SM-1 was reacted with KF to give the fluorinated pyridine compound 1. The N, N, N-trimethylammonium served as the leaving group and with the assistance of using microwave irradiation this nucleophilic substitution underwent very clean, very fast (completed in 1.5 min). Secondly, the tert-butyl protective group was removed with 90% TFA in dichloromethane to give the pyridine carboxylic acid 2. The microwave irradiation used in this step also greatly reduced the reaction time from traditional 30 min to 3 min. At the last, coupling the acid 2 with the dipeptide boronic acid D-Ala-boroPro SM-2 gave the target compound 3099D in 5 min. It was noted that the boronic acid group in SM-2 was unprotected, which allowed to skip the common de-protection step and thus further reduced the total reaction time.

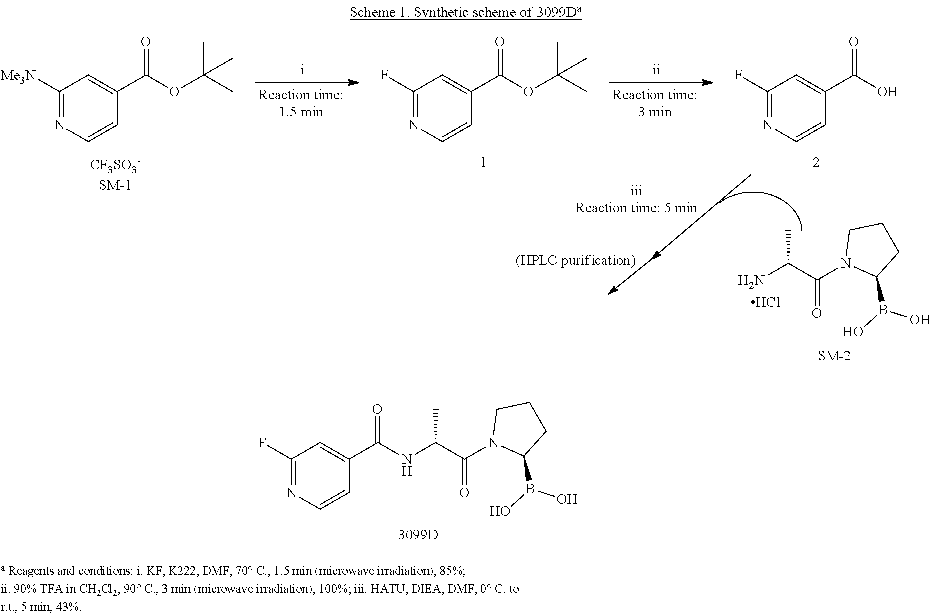

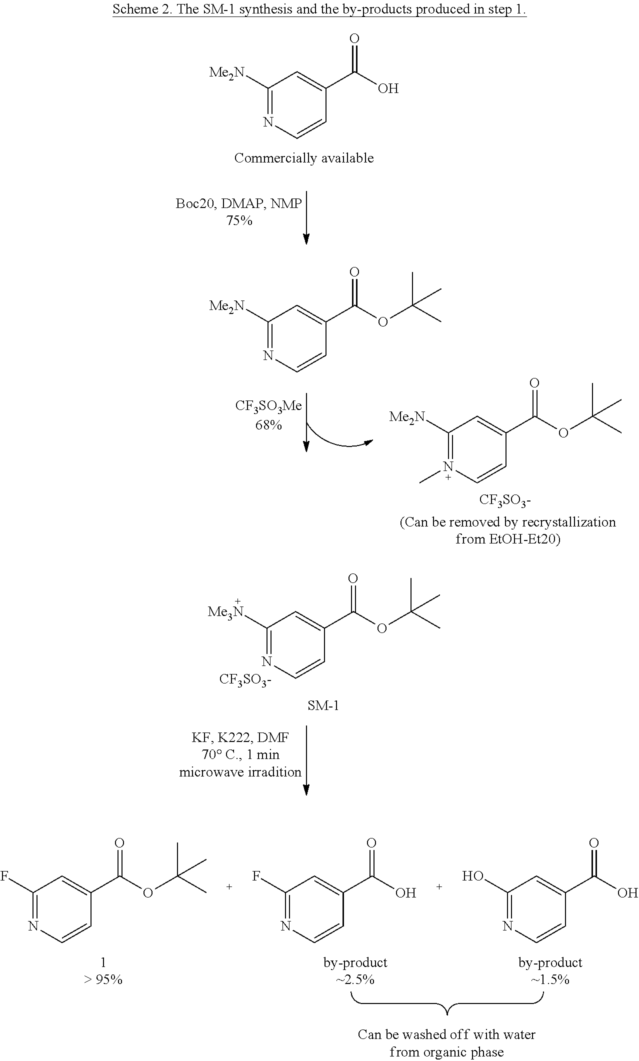

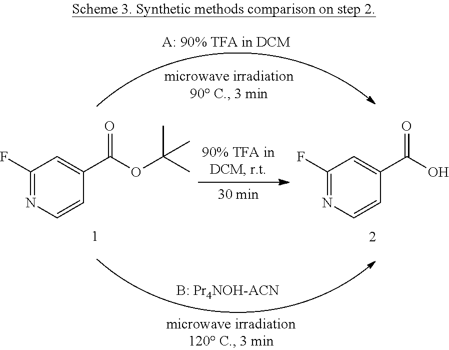

### (2) Method B.

There are only two steps involved in method B and the workup process can be simple. No workup is necessary except the HPLC purification at the final stage, all the by-products and impurities can be removed at the final stage by HPLC purification.

### FIG. 4. Synthetic Method of 3099D (Method B)^{a}

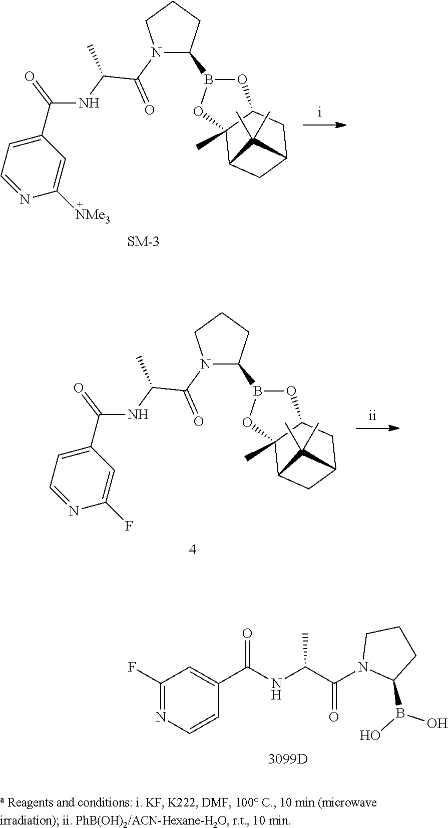

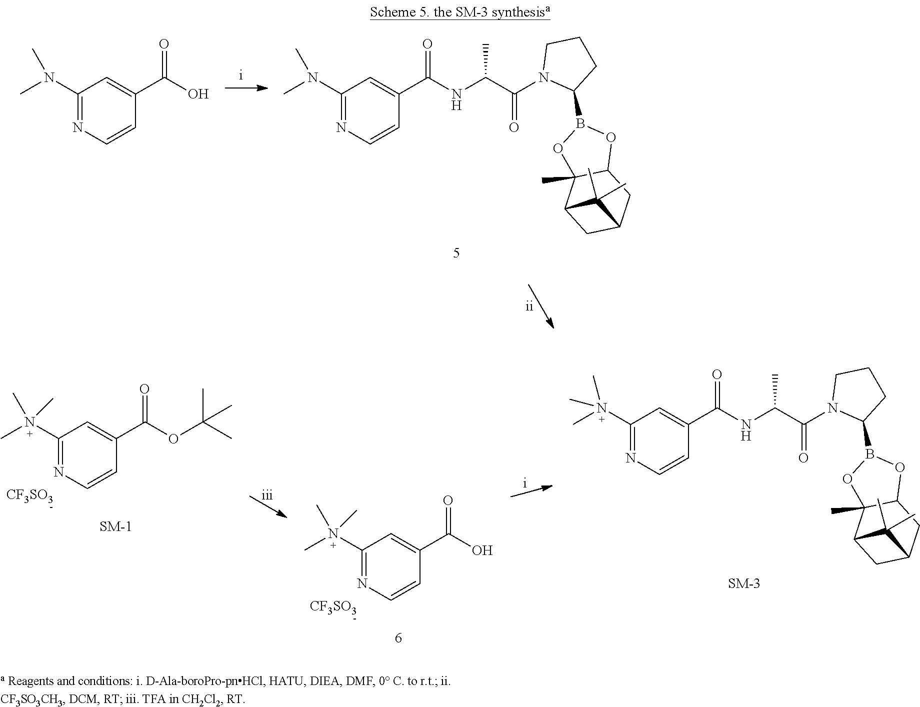

### (3) Method C.

There is only one steps involved in method C and all the by-products and impurities can be removed by a HPLC purification.

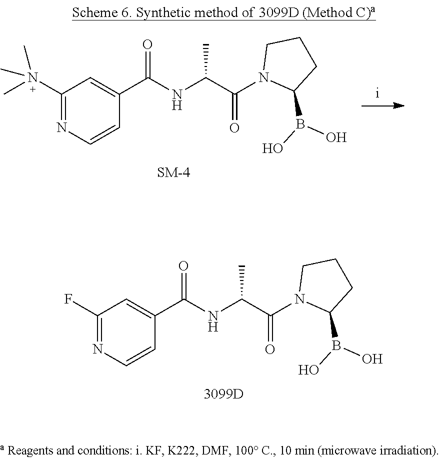

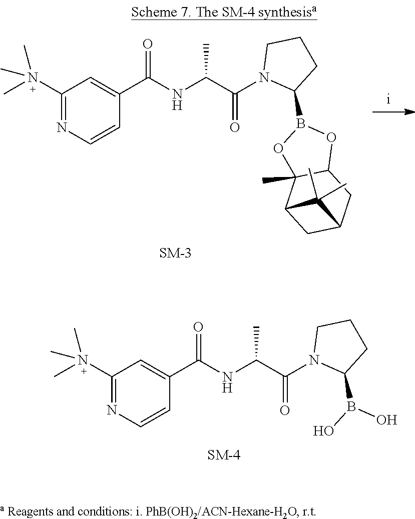

### (4) Method D (Minimalist Approach)

Due to the decreased nucleophilicity of [¹⁸F]Fluoride in aqueous solution, water has to be removed by azeotropic drying involving 2 to 3 cylces of MeCN addition in conventional method. Instead, the disclosure provides that [¹⁸F]Fluoride is directed eluted with an alcoholic solution containing the appropriate onium precursor, followed by evaporation of the alcohol and addition of a suitable solvent. The minimalist approach, “hot labling”, has several advantages that greatly improve its applicability. There is no longer a need for a base or other additives such as cryptand or bulky cations. Therefore, pH and temperature sensitive precursors can be applied and the overall synthesis time can be reduced.

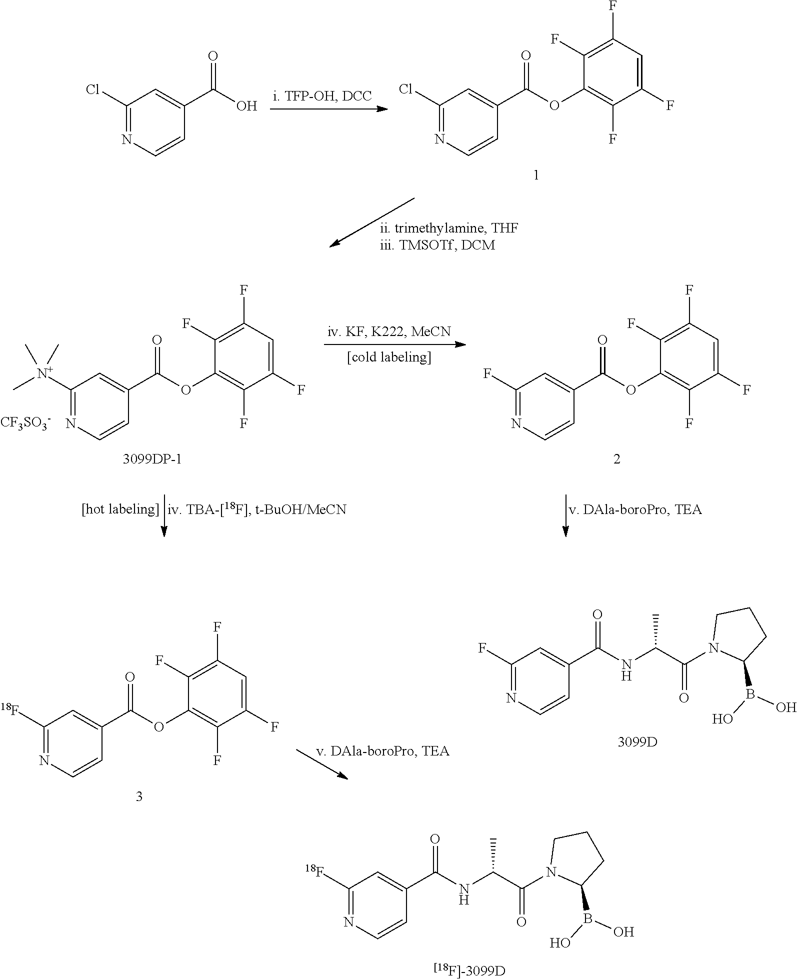

Hot labeling in the scheme above with [¹⁸F.]Fluoride, can be performed in the scheme below.

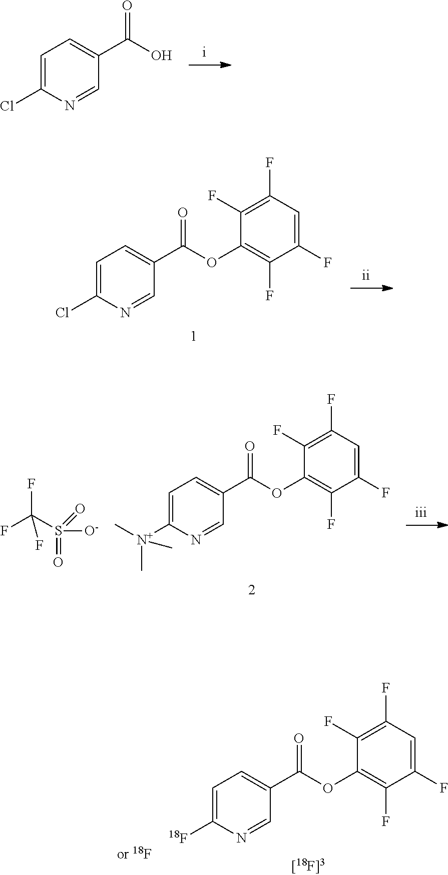 [SMILES: O=C(O)c1ccc(Cl)nc1 ; O=C(Oc1c(F)c(F)cc(F)c1F)c1ccc(Cl)nc1 ; O=S(=O)([O-])C(F)(F)F ; C[N+](C)(C)c1ccc(C(=O)Oc2c(F)c(F)cc(F)c2F)cn1 ; O=C(Oc1c(F)c(F)cc(F)c1F)c1ccc([18F])nc1]

Radiochemistry. Radiochemical syntheses of [¹⁸F] and [¹⁸F] were performed using the TRACERlab FX F-N(GE Medical Systems) with manual interventions when required. Isocratic reversed-phased preparative HPLC was run on a Phenomenex Luna C18(2) (150 mm×10 mm, 5 μm), flow 5 mL/min, using 21% solvent B for 20 min with UV detection at 254 nm using the in-built system of the TRACERlab FX F-N. Analytical HPLC was performed on an Agilent system (1100 series) with UV detection in series with a γ-detector (Bioscan flow-count)(see Supporting Information). Instant Imager (Packard BioScience) was used to measure the Radio-TLC scan. [¹⁸F]Fluoride was produced by a cyclotron (GE PETtrace 6) using ¹⁸O(p,n)18F nuclear reaction with a 16.5 MeV proton irradiation of an enriched [¹⁸O]H2O target.

Synthesis of N,N,N-Trimethyl-5-((2,3,5,6-tetrafluorophenoxy)-carbonyl)pyridin-2-aminium Trifluoromethanesulfonate (2). 6-Chloronicotinic acid 2,3,5,6-tetrafluorophenyl ester (1.0 g, 3.3 mmol) was dissolved in dry THF (15 mL). The solution was filtered into a 35 mL vial and capped with a rubber septum. A steady stream of trimethylamine gas at room temperature was passed through the filtrate under vigorous stirring, allowing excess trimethylamine to escape through a venting needle. After 5 min a white precipitate started to form and the reaction was allowed to proceed for 3 h while maintaining trimethylamine flow. The solid material was filtered off and washed with diethyl ether (100 mL) and cold dichloromethane (50 mL). The solid material (0.53 g, 1.5 mmol) (N,N,N-trimethyl-5-((2,3,5,6-tetra-fluorophenoxy)carbonyl)pyridin-2-aminium chloride) was sus-pended in dichloromethane (50 mL) under an argon atmosphere by means of ultrasonification. To the vigorously stirred suspen-sion was added trimethylsilyl trifluoromethanesulfonate (0.78 mL, 4.4 mmol) over 5 min. The solution was filtered, and volatile components were removed under reduced pressure. The dry residue was washed with diethyl ether (3×50 mL) and dried under vacuum, affording N,N,N-trimethyl-5-((2,3,5,6-tetra-fluorophenoxy)carbonyl)pyridin-2-aminium trifluoromethane-sulfonate (2) as a white fluffy solid (0.5 g, 32%). 1H NMR (500 MHz, CD₃CN): δ 9.34 (dd, J1=0.8 Hz, J2=2.3 Hz, 1H), δ 8.84 (dd, J1=2.3 Hz, J2=8.7 Hz, 1H), δ 8.07 (dd, J1=0.8 Hz, J2=8.7 Hz, 1H), δ 7.43 (tt, J1=7.3 Hz, J2=10.6 Hz, 1H), δ 3.60 (s, 9H). 19F NMR (470 MHz, CD3CN). δ-79.72 (s, 3F), δ-140.74 (m, 2F), δ-154.77 (m, 2F). Purity (HPLC): 98%, tR=1.76 min. HRMS-TOF (m/z): found, 329.1253 [M] [symbol] , calcd for C₁₅H₁₃F₄N₂O₂ 329.0913.

Synthesis of 6-Fluoronicotinic Acid 2,3,5,6-Tetrafluorophenyl Ester (3). A mixture of potassium fluoride (7.3 mg, 0.12 mmol) and Kryptofix 222 (59 mg, 0.16 mmol) in dry acetonitrile (1.0 mL) was stirred for 5 min. A solution of N,N,N-trimethyl-5-((2,3,5,6-tetrafluorophenoxy)carbonyl)pyridin-2-aminium trifluorome-thanesulfonate (2) (50 mg, 0.10 mmol) in dry acetonitrile (0.5 mL was added, and the resulting mixture was stirred for 15 min at room temperature. The reaction mixture was diluted with 2.0 mL water/0.1% TFA, filtered, and purified by reversed-phase preparative chromatography (Phenomenex Luna C18(2) column (250 mm×21.2 mm, 5 μm), flow rate of 10 mL/min, gradient of 40-80% solvent B over 60 min). The collected fractions were pooled, and acetonitrile was removed under reduced pressure. The aqueous phase was extracted with dichloromethane (3×10 mL). The combined organic phases were washed with water (10 mL), brine (10 mL) and dried (Na₂SO₄). The organic phase was removed in vacuo affording 6-fluoronicotinic acid 2,3,5,6-tetrafluorophenyl ester (3) as a waxy off-white solid (10 mg, 37%). ¹HNMR (500 MHz, CDC13): δ 9.10 (dt, Jₜ=0.5 Hz, Jd=2.5 Hz, 1H), δ 8.57 (ddd, J₁=2.5 Hz, J2=7.4 Hz, J3=8.6 Hz, 1H), δ 7.13 (ddd, J₁=0.5 Hz, J₂=3.0 Hz, J3=8.6 Hz, 1H), δ 7.09 (tt, J₁=7.1 Hz, J₂=9.9 Hz, 1H). ¹⁹F NMR (470 MHz, CDCl₃): δ−58.31 (dd, J₁=2.5 Hz, J₂=7.4 Hz 1F), δ-138.75 (m, 2F), δ-152.95 (m, 2F). Purity (HPLC): 99%, t_{R}=3.54 min. HRMS-TOF (m/z): found, 290.0249 [M [symbol] H] [symbol] , calcd for C₁₂H₄F₅NO₂ 290.0235.

### 2. Synthetic Method Suitable for the Preparation of [18F]-Labelled N-(2-Fluoroquinoline-4-Carbonyl)-D-Ala-boroPro (7073)

Compound 7073 was synthesized with the similar procedures as for 3099D.

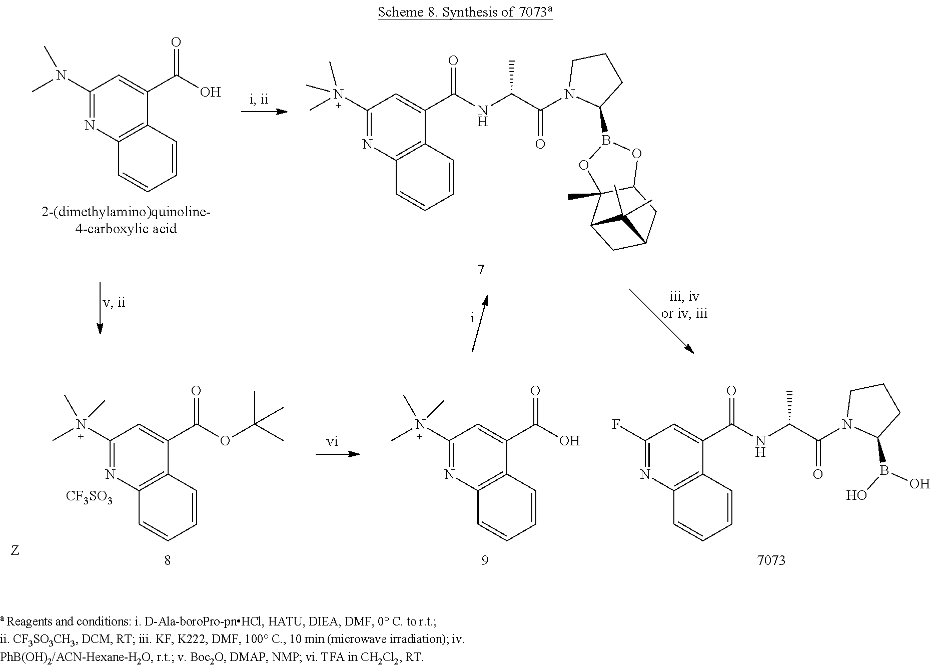

### Experimental Section

Reagents obtained from commercial sources were used without further purification. Synthesis of the L-boroPro-pn was performed using the previously described synthetic method (TS. J. Coutts etc. J. Med. Chem. 1996, 39, 2087-2094). All the target compounds were purified by RP-HPLC using Varian semi-preparative system with a Discovery C18 569226-U RP-HPLC column. The mobile phase for the semi-preparative HPLC was typically made by mixing water (0.1% TFA) with acetonitrile in gradient concentration. Mass spectra and HPLC retention times were recorded on a Hewlett Packard HP LC/MSD system with UV detector (monitoring at 215 nm), using an Eclipse Plus C18 RP-HPLC column (4.6×50 mm, 1.8 μm) with solvent gradient A) water (0.1% TFA) and B) acetonitrile at 0.5 mL/min. Unless otherwise noted, all HPLC retention times are given for an eluent gradient 2% B for the first 3 min, then from 2% to 98% B over 6 min, which was maintained for the next 6 min. NMR spectra were recorded on a Bruker Avance 300 MHz NMR spectrometer employing a 5 mm inverse multinuclear probe, unless otherwise noted. Chemical shifts were reported in parts per million (δ) relative to TMS (in CDCl₃) or DSS (in D₂O) for ¹H NMR.

### Synthesis of the Four Starting Materials:

For SM-1: 2-Dimethylamino-isonicotinic acid (500 mg, 3 mmol), DMAP (74 mg, 0.6 mmol) and Boc anhydride (1.74 g, 8 mmol) were dissolved in N-methyl-2-pyrrolidone (10 mL) and stirred at RT overnight. The reaction was quenched by the addition of a 0° C. solution of NaCl (0.2 g) and KH₂PO4 (0.2 g) in water (20 mL) and was extracted with dichloromethane. The organic phase was dried over anhydrous MgSO₄, filtered, evaporated and the crude product was purified by silica gel chromatography flash column (10% methanol in DCM) to give 2-N, N-dimethylaminopyridine-isonicotinate tert-butyl ester (500 mg, 75%).

To a stirred solution of the 2-N, N-dimethylamino-isonicotinate t-butyl ester (500 mg) in anhydrous DCM (10 mL), methyl trifluoromethanesulfonate (414 mg) was added dropwise. The reaction mixture was then stirred for 2 hrs at RT. Anhydrous t-butylmethylether was added, the white precipitate was collected and recrystallized with EtOH/Ether to give compound SM-1 (591 mg, 68%) as a pale-yellow powder. ¹H NMR (CDCl3): δ 1.65 (s, 9H), 3.82 (s, 9H), 8.09-8.12 (m, 1H), 8.28 (s, 1H), 8.76 (d, J=4.5 Hz, 1H). LC-MS (ESI⁺) m/z (rel intensity): 237.2 ([M+H]⁺, 100); tr=9.3 min.

For SM-2: To a stirred solution of N-Boc-D-Ala-OH (1.9 g, 10 mmol) in anhydrous DMF (40 mL) was added L-boroPro-pn.HCl (3.0 g, 10.5 mmol), HATU (4.0 g, 10.5 mmol) and DIEA (4.0 mL, 23 mmol) under ice-water bath cooling. The resulting mixture was stirred at room temperature for 2 hr. and then condensed in vacuo. The residue was dissolved with ethyl acetate (150 ml), washed sequentially by 0.1N KHSO₄ (3×40 mL), aq. NaHCO₃ (3×40 mL), brine (30 mL). The organic phase was dried over anhydrous MgSO₄, filtered, and evaporated in vacuo to give N-Boc-D-Ala-L-boroPro-pn which was purified by silica gel flash chromatography eluted with Ethyl Acetate/Hexane; and then added to a solution of 4N HCl in dioxane (30 mL) under ice-water cooling. The resulting mixture was stirred at room temperature for 2 hrs and then condensed in vacuo. The residue was co-evaporated with dichloromethane (3×30 mL) in vacuo to completely dry. Compound D-Ala-boroPro-pn.HCl was thus obtained as a white powder (3.3 g, 92% over two steps).

To a stirred solution of D-Ala-boroPro-pn.HCl (356 mg, 1 mmol) in water (5 mL) was added phenylboronic acid (132 mg, 1.1 mmol), acetonitrile (1 mL) and hexanes (10 mL). The resulting mixture was stirred at room temperature for 3 hrs and the separated aq. phase was washed by more hexanes. The aq. layer was condensed a little in vacuo and purified by semi-preparative HPLC. The combined fractions were lyophilized directly to give SM-2 as a white powder. LC-MS (ESI⁺) m, (rel intensity): 337.1 ([2×(M−H₂O)+H]⁺, 100); 168.9 ([M−H₂O+H]⁺, 45); tr=2.2 min.

For SM-3: To a stirred solution of 2-Dimethylamino-isonicotinic acid (0.83 g, 5 mmol) in anhydrous DMF (20 mL) was added D-Ala-boroPro-pn.HCl (1.95 g, 5.5 mmol), HATU (2.0 g, 5.3 mmol) and DIEA (2.0 mL, 11.5 mmol) under ice-water bath cooling. The resulting mixture was stirred at room temperature for 2 hrs and then condensed in vacuo. The residue was dissolved with dichloromethane (100 mL), washed sequentially by aq. NaHCO₃ (3×20 mL), brine (20 mL). The organic phase was dried over anhydrous MgSO₄, filtered, and evaporated in vacuo to give N-Boc-D-Ala-L-boroPro-pn which was purified by silica gel flash chromatography eluted with 0-5% of MeOH in dichloromethane to give 2.3 g of the Compound 5 as colorless oil. LC-MS (ESI⁺) m/z (rel intensity). 468.2 ([M+H]⁺, 45), tr=8.8 min.

To a stirred solution of the Compound 5 (1.6 g) in anhydrous DCM (10 mL), methyl trifluoromethanesulfonate (414 mg) was added dropwise. The reaction mixture was stirred at RT overnight and then condensed in vacuo to give compound SM-3 (2.0 g) as a pale-yellow oil. LC-MS (ESI⁺) m/z (rel intensity): 483.2 ([M+H]⁺, 100); tr=8.9 min.

For SM-4: To a stirred solution of compound SM-4 (500 mg) in water (10 mL) was added phenylboronic acid (120 mg), acetonitrile (3 mL) and hexanes (20 mL). The resulting mixture was stirred at room temperature for 2 hrs and the separated aq. phase was washed by more hexanes. The aq. layer was condensed a little in vacuo and purified by semi-preparative HPLC. The combined fractions were lyophilized directly to give SM-4 as a white powder. LC-MS (ESI⁺) m/z (rel intensity): 349.8 ([M+H]+, 100); tr=7.4 min.

### Synthesis of Compound 1

Compound SM-1 (25 mg, 0.065 mmol), K222 (4,7,13,16,21,24-Hexaoxa-1,10-diazabicyclo [8.8.8]hexacosane, 50 mg, 0.13 mmol) and KF (spray-dried, 75 mg, 1.3 mmol) were mixed together in anhydrous DMF (0.3 mL) under Ar protection in a 10-mL Pyrex glass vial (CEM). The reaction was carried out in a closed vessel using a microwave synthesis reactor (CEM Discover Labmate) under continuous stirring. The microwave irradiation power was set for 200 W; reaction temperature was 70° C. and the hold time was 1.5 min. After cooled down the reaction mixture was diluted with ethyl ether (30 mL), washed sequentially with 5% citric acid (4×10 mL), water (10 mL), aq. NaCl (10 mL). The organic phase was dried with MgSO₄, filtered. The solvent was removed in vacuo and the obtained crude compound 1 (11 mg, 85%) was used directly in the next step. LC-MS (ESI⁺) m/z (rel intensity): 198.0 ([M+H]⁺, 100); 142.0 (38); tr=10.7 min.

### Synthesis of Compound 2

Crude compound 1 (11 mg) was dissolved into a solution of 90% TFA in dichloromethane (0.5 mL) under Ar protection in a 10-mL Pyrex glass vial (CEM). The reaction was carried out in a closed vessel using a microwave synthesis reactor (CEM Discover Labmate) under continuous stirring. The microwave irradiation power was set for 200 W; reaction temperature was 90° C. and the hold time was 3 min. After completion, the solvent was removed in vacuo. The residue was co-evaporated with dichloromethane (3×5 mL) in vacuo to give crude compound 2 as a TFA salt (14 mg, 100%) which was used directly in the next step. LC-MS (ESI⁺) m/z (rel intensity): 142.0 ([M+H]⁺, 100); tr=10.7 min.

### Synthesis of 3099D

Method A. To a stirred solution of crude compound 2 (14 mg, 0.055 mmol) in anhydrous DMF (0.6 mL) was added N, N-diisopropylethylamine (DIEA, 30 IL, 0.165 mmol) (The pH should be approximately 9-10 after DIEA addition: more DIEA may be needed if the residual TFA was not removed completely in crude compound 2) and HATU (25 mg, 0.066 mmol) under a cooling water bath. The solution was stirred for 1 min and then SM-2 (15 mg, 0.066 mmol), an additional portion of DIEA (10 μL, 0.055 mmol) were added. The cooling bath was removed, and the resulting mixture was stirred at room temperature for 5 min. The solvent was then removed in vacuo under 30° C. (to remove most of DMF and DIEA). The residue was dissolved in 0.1% TFA water solution (2 mL) (a little methanol may be added to improve the solubility) and was purified by semi-preparative HPLC (Mobile phase A: 0.1% TFA in water; Mobile phase B: 0.08% TFA in acetonitrile). The desired fraction was collected and dried in vacuo to give 3099D TFA salt (10 mg, 43%) as a white powder. ¹H NMR (D₂O): δ 1.38 (d, J=7.1 Hz, 3H), 1.61-1.66 (m, 1H), 1.94-2.10 (m, 3H), 2.91-2.97 (m, 1H), 3.59-3.65 (m, 2H), 4.75 (q, J=7.1 Hz, 1H), 7.36 (s, 1H), 7.57 (d, J=5.3 Hz, 1H), 8.27 (d, J=5.3 Hz, 1H). LC-MS (ESI⁺) m/z(rel intensity): 292.1 ([M−H₂O+H]⁺, 100); tr=5.8 min (Gradient: 0-3 min, 10% B; 3-9 min, 10-50% B; 9-12 min, 98% B).

Method B. Compound 4 was synthesized similar with the synthesis of Compound 1 from SM-1, except reacted at 100° C. for 10 min; and then was removed the pinanediol group with the similar procedure with the synthesis of SM-2 from D-Ala-boroPro-pn, except the reaction time was 10 mins, to give 3099D.

Method C. The Compound SM-4 was reacted with KF by the similar procedure with the synthesis of Compound 1 from SM-1, except reacted at 100° C. for 10 min, to give 3099D.

### Example 2: Biological Activity of Exemplary Compounds of Formula I of the Disclosure Biological Materials

The peptide libraries XPYSWS-NH₂, Ac-XPYSWS-NH₂, GXYSWS-NH₂, and Ac-GXYSWS-NH₂, where X represents all natural amino acids except cysteine were synthesized by the Tufts University Core Facility. The DPPIV enzyme used for the specificity studies was previously purified by our laboratory at Tufts University from human placenta, and the purified human FAP enzyme used for these studies was kindly provided by Syrxx, Inc. For the in vitro IC₅₀ determination assays, recombinant human DPPIV, DPP9, FAP, and PREP were purchased from R&D Systems, and DPP8 was from Biomol International. Buffer systems used were A (25 mM Tris, pH 8.0), B (50 mM Tris, pH 7.5), C (50 mM Tris, 140 mM NaCl, pH 7.5), D (25 mM Tris, 250 mM NaCl, pH 7.5), and E (20 mM Tris, 20 mM KCl, pH 7.4). Fluorogenic substrates were Gly-Pro-AMC, Z-Gly-Pro-AMC, or Suc-Gly-Pro-AMC purchased from Bachem or an N-terminally blocked FAP specific substrate.⁴² The cell culture medium was RPMI 1640 without phenol red and supplemented with 2 mM-glutamine, 10 mM HEPES, 1 mM sodium pyruvate, 4500 mg/L glucose, 100 IU/mL penicillin, and 100 μg/mL streptomycin.

### Substrate Specificity Assay

Peptide libraries (0.21 mM) were incubated for 24 h with 1 nM FAP in buffer E at 37° C. The reaction was quenched by the addition of 1.2 N HCl. The samples were analyzed by reverse-phase HPLC-MS on a Thermo Finnigan LCQ Duo, quantifying the peaks in the resulting base peak chromatograms. Relative cleavage values were determined by comparing the postquench abundance of intact peptides to those in the initial library.

### In Vitro Enzyme IC₅₀ Assays

Enzymatic activity of DPPIV, DPP8, DPP9, FAP, and PREP was measured at 25° C. on a Molecular Devices M2^{e} multidetection microtiter plate reader, monitoring the fluorescence at an excitation wavelength of 380 nm and an emission wavelength of 460 nm. The substrate was either H-Gly-Pro-AMC for the DPPIV, DPP8, and DPP9 assays or Z-Gly-Pro-AMC for the FAP and PREP assays. The reaction mixture contained 25 μM substrate, enzyme, buffer

A (DPPIV and DPP9), buffer B (DPP8), buffer C (FAP), or buffer D (PREP) and a suitable amount of inhibitor (ranging between 10⁻⁴ and 10⁻¹¹M) in a total volume of 210 μL. The final enzyme concentrations were 0.1, 0.8, 0.4, 1.2, and 0.6 nM for DPPIV, DPP8, DPP9,

FAP, and PREP, respectively. The IC₅₀ value is defined as the concentration of inhibitor required to reduce the enzyme activity by 50% after a 10 min preincubation with the enzyme at 25° C. prior to addition of the substrate. Inhibitor stock solutions (100 mM) were prepared in either a pH 2.0 HCl solution for compounds 1 and 20 or DMSO. Those prepared in pH 2.0 solution were preincubated at 25° C. for 4 h prior to dilution. Immediately prior to the commencement of the experiment, the 100 mM stocks were further diluted to 10⁻³M in the appropriate assay buffer, from which 1:10 serial dilutions were prepared. All inhibitors were tested in triplicate.

### Inhibition of Cellular Enzyme Activity in HEK293 Cells

Mock or murine FAP (mFAP) transfected HEK293 cells were kindly provided by Jonathan Cheng from the Fox Chase Cancer Center. The cells were equilibrated in RPMI 1640 at a density of 25 000 cells/well, treated with 1 μM 2, 6, 22, or RPMI 1640 alone, and allowed to incubate for 15 min at 37° C. The substrate was 25 μM Z-Gly-Pro-AMC. The fluorescence was measured at 37° C., monitoring an excitation wavelength of 380 nm and an emission wavelength of 460 nm. The percent activity was determined versus the RPMI 1640 control.

### In Vitro Mixed FAP and PREP Inhibition Assay

Enzymatic activity of 1 nM FAP combined with 0.01-100 nM PREP was measured at 25° C., monitoring the fluorescence at an excitation wavelength of 380 nm and an emission wavelength of 460 nm. The substrate was the N-terminally blocked FAP specific substrate, so as to be able to differentiate FAP activity in the presence of PREP. The final reaction mixture contained 25 μM substrate, enzyme, a 1:1 mixture of buffer C and buffer D, and either 36 nM or 1 μM 6 in a total volume of 210 μL. The enzymes were allowed to preincubate with the inhibitor for 10 min at room temperature prior to addition of the substrate. The percent of FAP activity was determined based on a simultaneously run control for each condition, which lacked treatment with the inhibitor.

### Measurement of FAP and PREP Activity in HEK293 Cells

Both mock and mFAP transfected HEK293 cells were equilibrated in RPMI 1640 at a density of 25 000 cells/well. The mock transfected cells were treated with 25 μM Z-GP- AMC, and the mFAP transfected cells were treated with 50 μM N-terminally blocked FAP specific inhibitor in a total volume of 200 μL/well. The fluorescence was measured at 37° C., monitoring an excitation wavelength of 380 nm and an emission wavelength of 460 nm. To calculate the relative cellular PREP and FAP concentrations, standard activity curves were prepared for each using varying concentrations of purified enzyme and Z-GP-AMC for PREP and the N-terminally blocked FAP specific substrate for FAP. The relative concentration of active enzyme associated with the cells was calculated via interpolation from the standard curves and the measured cellular activity.

| | | | PREP | | | |
|---|---|---|---|---|---|---|
| | FAP Inhibition | | Inhibition | | FAP | |
| | (IC₅₀, nM) | | (IC₅₀, nM) | | Selectivityª | |
| Structure | pH2.0 | pH7.5 | pH2.0 | pH7.5 | pH2.0 | pH7.5 |
| 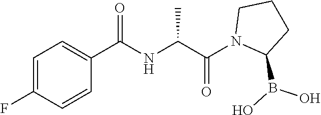 [SMILES: C[C@@H](NC(=O)c1ccc(F)cc1)C(=O)N1CCC[C@H]1B(O)O] | 220 | 260 | 4100 | 5700 | 19 | 22 |
| 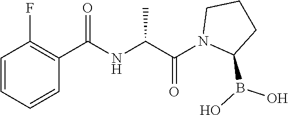 [SMILES: C[C@@H](NC(=O)c1ccccc1F)C(=O)N1CCC[C@H]1B(O)O] | 390 | 650 | 2200 | 3100 | 5.6 | 4.8 |
| 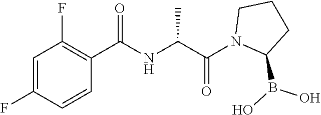 [SMILES: C[C@@H](NC(=O)c1ccc(F)cc1F)C(=O)N1CCC[C@H]1B(O)O] | 690 | 390 | 790 | 1400 | 1.1 | 3.6 |
| 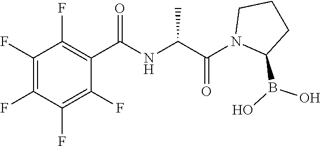 [SMILES: C[C@@H](NC(=O)c1c(F)c(F)c(F)c(F)c1F)C(=O)N1CCC[C@H]1B(O)O] | 450 | 330 | 1200 | 1500 | 2.7 | 4.5 |
| 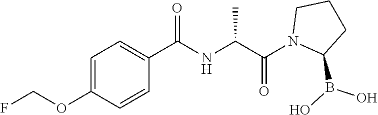 [SMILES: C[C@@H](NC(=O)c1ccc(OCF)cc1)C(=O)N1CCC[C@H]1B(O)O] | 240 | 240 | 2000 | 1900 | 8.3 | 7.9 |
| 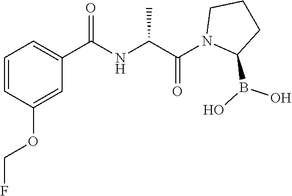 [SMILES: C[C@@H](NC(=O)c1cccc(OCF)c1)C(=O)N1CCC[C@H]1B(O)O] | 130 | 130 | 48 | 53 | 0.37 | 0.41 |
| 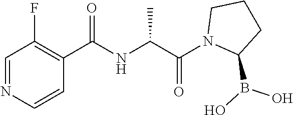 [SMILES: C[C@@H](NC(=O)c1ccncc1F)C(=O)N1CCC[C@H]1B(O)O] | 14 | 15 | 1340 | 1570 | 96 | 105 |
| 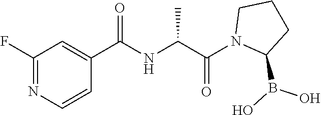 [SMILES: C[C@@H](NC(=O)c1ccnc(F)c1)C(=O)N1CCC[C@H]1B(O)O] | 16.7 | 15.6 | 2340 | 2110 | 140 | 135 |
| 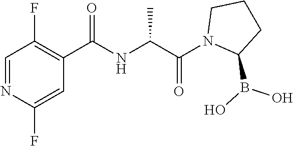 [SMILES: C[C@@H](NC(=O)c1cc(F)ncc1F)C(=O)N1CCC[C@H]1B(O)O] | 140 | 140 | 2000 | 2300 | 14 | 16 |
| 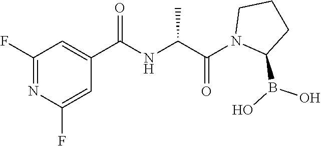 [SMILES: C[C@@H](NC(=O)c1cc(F)nc(F)c1)C(=O)N1CCC[C@H]1B(O)O] | 100 | 130 | 2300 | 2500 | 23 | 19 |
| 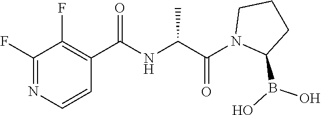 [SMILES: C[C@@H](NC(=O)c1ccnc(F)c1F)C(=O)N1CCC[C@H]1B(O)O] | 130 | 150 | 1900 | 2200 | 15 | 15 |

ªThe FAP selectivity is equal to IC₅₀(PREP)/IC₅₀(FAP). IC₅₀ values are expressed with ±SEM.

### Example 3: Biological Activity of Exemplary Compounds of Formula XI (FAP Inhibition)

Biological Activity Assays of FAP Inhibition for these Compounds Below were Performed as Described in Example 2.

| | FAP |
|---|---|
| | Inhibition |
| Structure | (IC₅₀, nM) |
| 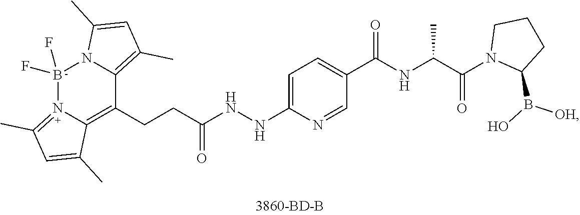 | 0.4 |
| 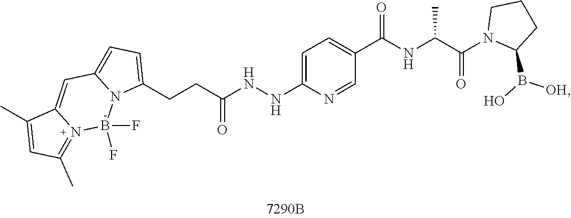 | 13.9 |

### Example 4: Treatment Protocol

A human patient with treatment resistant prostate cancer is selected for therapy. [¹⁸F]-labelled N-(2-Fluoropyrid-ine-4-Carbonyl)-D-Ala-boroPro (3099D compound) in a sterile aqueous solution is administered by intravenous injection to the patient. Single-dose vials are used that contain the 3099D compound. The 3099D may be administered over multiple such as 2, 3, 4 or more four treatment cycles, with one injection per treatment cycle.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A compound having a structure represented by formula I or a pharmaceutically acceptable salt thereof:
Z-A-(FAPX)ₙ₁ (I)
wherein,
FAPx is FAPi or FAPb;
FAPi is a compound which covalently binds to FAP;
FAPb is a compound which non-covalently binds to FAP;
A is cycloalkyl, aryl, heteroaryl, or heterocyclyl;
Z is a radioactive halogen isotope or trialkylammonium; and
n₁ is 1-6.

2. A compound having a structure represented by formula XI or a pharmaceutically acceptable salt thereof:
(Z)_{z1}-A₂-L₂-A₁-(FAPx)ₙ₁ (XI)
wherein,
FAPx is FAPi or FAPb;
FAPi is a compound which covalently binds to FAP;
FAPb is a compound which non-covalently binds to FAP;
Each A₁, and A₂ is independently cycloalkyl, aryl, heteroaryl, or heterocyclyl;
L² is a bond or a covalent linker (e.g., a substituted or unsubstituted C₁-C₁₂ alkylene, or a substituted or unsubstituted 2 to 12 membered heteroalkylene);
Z is a radioactive halogen isotope or trialkylammonium;
each z1 is independently 1-3; and
n₁ is 1-6.

3. A compound having a structure represented by formula XXI or a pharmaceutically acceptable salt thereof: 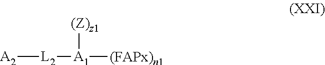
wherein,
FAPx is FAPi or FAPb;
FAPi is a compound which covalently binds to FAP;
FAPb is a compound which non-covalently binds to FAP;
Each A₁, and A₂ is independently cycloalkyl, aryl, heteroaryl, or heterocyclyl;
L² is a bond or a covalent linker (e.g., a substituted or unsubstituted C₁-C₁₂ alkylene, or a substituted or unsubstituted 2 to 12 membered heteroalkylene);
Z is a radioactive halogen isotope or trialkylammonium;
each z1 is independently 1-3; and
n₁ is 1-6.

4. The compound of any one of claims 1-3, wherein FAN is FAPi.

5. The compound of any one of claims 1-4, wherein FAPi covalently binds to a side chain of an amino acid in an active site of FAP.

6. The compound of any one of claims 1-5, wherein FAPi is a reversible FAP inhibitor.

7. The compound of any one of claims 1-5, wherein FAPi is an irreversible FAP inhibitor.

8. The compound of any one of claims 1-7, wherein FAPi comprises a moiety which has a Kᵢ for FAP that is at least 10× smaller as compared to the Kᵢ of prolyl endopeptidase for FAP (EC 3.4.21.26; PREP).

9. The compound of any one of claims 1-8, wherein FAPi comprises a moiety which has a Kᵢ for FAP that is at least 100× smaller as compared to the Kᵢ of prolyl endopeptidase for FAP (EC 3.4.21.26; PREP).

10. The compound of any one of claims 1-9, wherein FAPi comprises a moiety which has a Kᵢ for FAP that is at least 1,000× smaller as compared to the Kᵢ of prolyl endopeptidase for FAP (EC 3.4.21.26; PREP).

11. The compound of any one of claims 1-10, wherein FAPi comprises a moiety which has a Kᵢ for FAP that is at least 5,000× smaller as compared to the Kᵢ of prolyl endopeptidase for FAP (EC 3.4.21.26; PREP).

12. The compound of any one of claims 1-11, wherein FAPi comprises a moiety which has a Kᵢ for FAP that is at least 10,000× smaller as compared to the Kᵢ of prolyl endopeptidase for FAP (EC 3.4.21.26; PREP).

13. The compound of any one of claims 1-12, wherein FAPi comprises a moiety which has a Kᵢ for FAP that is less than 10⁻⁶M.

14. The compound of any one of claims 1-13, wherein FAPi comprises a moiety which has a Kᵢ for FAP that is less than 10⁻⁷M.

15. The compound of any one of claims 1-14, wherein FAPi comprises a moiety which has a Kᵢ for FAP that is less than 10⁻⁸M.

16. The compound of any one of claims 1-15, wherein FAPi comprises a moiety which has a Kᵢ for FAP that is less than 10⁻⁹M.

17. The compound of any one of claims 1-16, wherein FAPi comprises a moiety which has a Kᵢ for FAP that is less than 10⁻¹M.

18. The compound of any one of claims 1-3, wherein FAN is FAP_{b}.

19. The compound of claim 18, wherein FAP_{b} forms a complex with FAP.

20. The compound of claim 18 or 19, wherein FAP_{b} comprises a moiety which has a K for FAP that is less than 10⁻⁶M.

21. The compound of any one of claims 18-20, wherein FAP_{b} comprises a moiety which has a K_{d} for FAP that is less than 10⁻⁷M.

22. The compound of any one of claims 18-21, wherein FAP_{b} comprises a moiety which has a K_{d} for FAP that is less than 10⁻⁸M.

23. The compound of any one of claims 18-22, wherein FAP_{b} comprises a moiety which has a K_{d} for FAP that is less than 10⁻⁹M.

24. The compound of any one of claims 19-23, wherein FAP_{b} comprises a moiety which has a K_{d} for FAP that is less than 10⁻¹⁰M.

25. The compound of any one of claims 1-24, wherein n₁ is 1.

26. The compound of claim 25, wherein the compound has a structure represented by formula II or a pharmaceutically acceptable salt thereof: 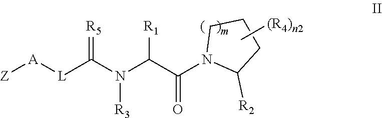
wherein,
A is cycloalkyl, aryl, heteroaryl, or heterocyclyl;
Z is a radioactive halogen isotope or a trialkylammonium;
L is a bond or a covalent linker;
R₁ is H or alkyl;
R₂ is a moiety which covalently binds to a side chain of an amino acid in an active site of FAP;
R₃ is H or alkyl;
R₄ is alkyl, hydroxyl, amino, or halo;
R₅ is O or S;
m is 1-3; and
n₂ is 0-7.

27. The compound of claim 25, wherein the compound has a structure represented by formula (XII), or a pharmaceutically acceptable salt thereof: 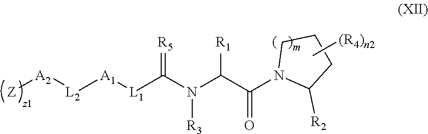
wherein,
each A₁, and A₂ is independently is cycloalkyl, aryl, heteroaryl, or heterocyclyl;
Z is a radioactive halogen isotope or a trialkylammonium;
each L₁, and L₂ is independently a bond, a substituted or unsubstituted C₁-C₁₂ alkylene, or a substituted or unsubstituted 2 to 12 membered heteroalkylene;
R₁ is H or alkyl;
R₂ is a moiety which covalently binds to a side chain of an amino acid in an active site of FAP;
R₃ is H or alkyl;
R₄ is alkyl, hydroxyl, amino, or halo;
R₅ is O or S;
m is 1-3;
z is 1-3; and
n₂ is 0-7.

28. The compound of claim 25, wherein the compound has a structure represented by formula (XXII), or a pharmaceutically acceptable salt thereof: 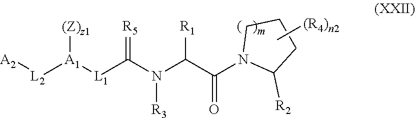
wherein,
each A₁, and A₂ is independently is cycloalkyl, aryl, heteroaryl, or heterocyclyl;
Z is a radioactive halogen isotope or a trialkylammonium;
each L₁, and L₂ is independently a bond, a substituted or unsubstituted C₁-C₁₂ alkylene, or a substituted or unsubstituted 2 to 12 membered heteroalkylene;
R₁ is H or alkyl;
R₂ is a moiety which covalently binds to a side chain of an amino acid in an active site of FAP;
R₃ is H or alkyl;
R₄ is alkyl, hydroxyl, amino, or halo;
R₅ is O or S;
m is 1-3;
z1 is 1-3; and
n₂ is 0-7.

29. The compound of claim 26, wherein the compound has a structure represented by formula (XIII), or a pharmaceutically acceptable salt thereof: 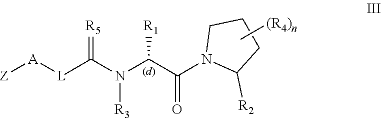
wherein,
A is cycloalkyl, aryl, heteroaryl, or heterocyclyl;
Z is a radioactive halogen isotope or a trialkylammonium;
L is a bond or a covalent linker;
R₁ is H or alkyl;
R₂ is a moiety which covalently binds to a side chain of an amino acid in an active site of FAP;
R₃ is H or alkyl;
R₄ is alkyl, hydroxyl, amino, or halo;
R₅ is O or S; and
n is 0-7.

30. The compound of claim 27, wherein the compound has a structure represented by formula (XIII), or a pharmaceutically acceptable salt thereof: 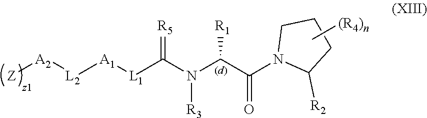
wherein,
each A₁, and A₂ is independently cycloalkyl, aryl, heteroaryl, or heterocyclyl;
Z is a radioactive halogen isotope or a trialkylammonium;
each L₁, and L₂ is independently a bond, a substituted or unsubstituted C₁-C₁₂ alkylene, or a substituted or unsubstituted 2 to 12 membered heteroalkylene;
R₁ is H or alkyl;
R₂ is a moiety which covalently binds to a side chain of an amino acid in an active site of FAP;
R₃ is H or alkyl;
R₄ is alkyl, hydroxyl, amino, or halo;
R₅ is O or S;
z1 is 1-3; and
n is 0-7.

31. The compound of claim 28, wherein the compound has a structure represented by formula (XXIII), or a pharmaceutically acceptable salt thereof: 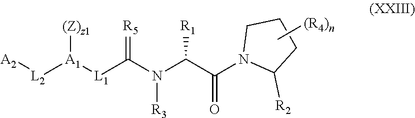
wherein,
each A₁, and A₂ is independently cycloalkyl, aryl, heteroaryl, or heterocyclyl;
Z is a radioactive halogen isotope or a trialkylammonium;
each L₁, and L₂ is independently a bond, a substituted or unsubstituted C₁-C₁₂ alkylene, or a substituted or unsubstituted 2 to 12 membered heteroalkylene;
R₁ is H or alkyl;
R₂ is a moiety which covalently binds to a side chain of an amino acid in an active site of FAP;
R₃ is H or alkyl;
R₄ is alkyl, hydroxyl, amino, or halo;
R₅ is O or S;
z1 is 1-3; and
n is 0-7.

32. The compound of any one of claims 26-31, wherein R₁ is (C₁-C₆)alkyl (e.g., methyl or ethyl).

33. The compound of any one of claims 26-31, wherein R₁ is H, (C₁-C₆)alkyl (e.g., methyl or ethyl), or (C₁-C₆)alkyl substituted with —OH.

34. The compound of any one of claims 26-33, wherein R₂ is B(Y¹)(Y²), CN, or formyl; wherein Y¹ and Y² are each hydroxyl; or Y¹ and Y² together with the boron atom to which they are attached combine to form a moiety which is hydrolysable to a boronic acid.

35. The compound of claim 34, wherein Y¹ and Y² together with the boron atom to which they are attached combine to form a 5- to 8-membered ring.

36. The compound of any one of claims 26 to 33, wherein R₂ is B(OH)₂.

37. The compound of any one of claims 26-36, wherein R₃ is (C₁-C₆)alkyl.

38. The compound of any one of claims 26-36, wherein R₃ is H.

39. The compound of any one of claims 26-38, wherein R₃ is (C₁-C₆)alkyl.

40. The compound of any one of claims 26-38, wherein R₄ is halo (e.g., fluorine).

41. The compound of any one of claims 26-40, wherein R₅ is O.

42. The compound of any one of claims 26-41, wherein n₂ is 2.

43. The compound of any one of claims 26-42, wherein n₂ is 0.

44. The compound of claim 26, wherein the compound has a structure represented by formula IV-A, or a pharmaceutically acceptable salt thereof: 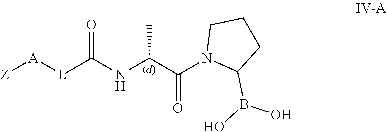
wherein,
A is cycloalkyl, aryl, heteroaryl, or heterocyclyl;
Z is a radioactive halogen isotope or a trialkylammonium; and
L is a bond or a covalent linker.

45. The compound of claim 27, wherein the compound has a structure represented by formula (XIV-A), or a pharmaceutically acceptable salt thereof: 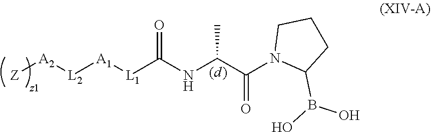
wherein,
each A₁, and A₂ is independently cycloalkyl, aryl, heteroaryl, or heterocyclyl;
Z is a radioactive halogen isotope or a trialkylammonium; and
each L₁, and L₂ is independently a bond or a covalent linker.

46. The compound of claim 28, wherein the compound has a structure represented by formula (XXIV-A), or a pharmaceutically acceptable salt thereof: 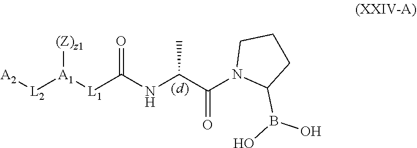
wherein,
each A₁, and A₂ is independently cycloalkyl, aryl, heteroaryl, or heterocyclyl;
Z is a radioactive halogen isotope or a trialkylammonium; and
each L₁, and L₂ is independently a bond or a covalent linker.

47. The compound of claim 26, wherein the compound has a structure represented by formula (IV-B) or a pharmaceutically acceptable salt thereof: 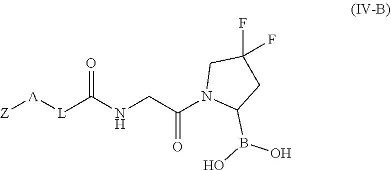
wherein,
A is cycloalkyl, aryl, heteroaryl, or heterocyclyl;
Z is a radioactive halogen isotope or a trialkylammonium; and
L is a bond or a covalent linker.

48. The compound of any one of claims 1-24, wherein the compound has a structure represented by formula V or a pharmaceutically acceptable salt thereof: 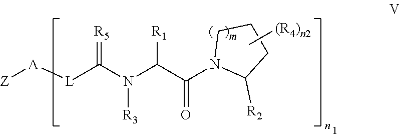
wherein,
A is cycloalkyl, aryl, heteroaryl, or heterocyclyl;
Z is a radioactive halogen isotope or a trialkylammonium;
L is a bond or a covalent linker;
R₁ is H or alkyl;
R₂ is a moiety which covalently binds to a side chain of an amino acid in an active site of FAP;
R₃ is H or alkyl;
R₄ is alkyl, hydroxyl, amino, or halo;
R₅ is O or S;
m is 1-3;
n₁ is 2-6; and
n₂ is 0-7.

49. The compound of any one of claims 1-24, wherein the compound has a structure represented by formula VI or a pharmaceutically acceptable salt thereof: 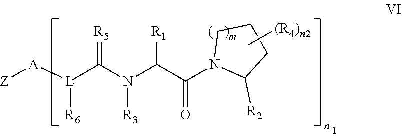
wherein,
A is cycloalkyl, aryl, heteroaryl, or heterocyclyl;
Z is a radioactive halogen isotope or a trialkylammonium;
L is a bond or a covalent linker;
R₁ is H or alkyl;
R₂ is a moiety which covalently binds to a side chain of an amino acid in an active site of FAP;
R₃ is H or alkyl;
R₄ is alkyl, hydroxyl, amino, or halo;
R₅ is O or S;
R_{b} is a moiety which modifies the pharmacokinetics or bio-distribution of the compound;
m is 1-3;
n₁ is 2-6; and
n₂ is 0-7.

50. The compound of any one of claims 1-24, wherein the compound has a structure represented by formula VII or a pharmaceutically acceptable salt thereof: 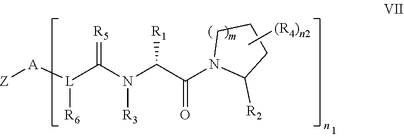
wherein,
A is cycloalkyl, aryl, heteroaryl, or heterocyclyl;
Z is a radioactive halogen isotope or a trialkylammonium;
L is a bond or a covalent linker;
R₁ is H or alkyl;
R₂ is a moiety which covalently binds to a side chain of an amino acid in an active site of FAP;
R₃ is H or alkyl;
R₄ is alkyl, hydroxyl, amino, or halo;
R₅ is O or S;
R₆ is a moiety which modifies the pharmacokinetics or bio-distribution of the compound;
m is 1-3;
n₁ is 2-6; and
n₂ is 0-7.

51. The compound of any one of claims 48-50, wherein R₁ is (C₁-C₆)alkyl (e.g., methyl or ethyl).

52. The compound of any one of claims 48-50, wherein R₁ is H, (C₁-C₆)alkyl (e.g., methyl or ethyl), or (C₁-C₆)alkyl substituted with —OH.

53. The compound of any one of claims 48-52, wherein R₂ is B(Y¹)(Y²), CN, or formyl; wherein Y¹ and Y² are each hydroxyl; or Y¹ and Y² together with the boron atom to which they are attached combine to form a moiety which is hydrolysable to a boronic acid.

54. The compound of claim 53, wherein Y¹ and Y² together with the boron atom to which they are attached combine to form a 5- to 8-membered ring.

55. The compound of any one of claims 48-52, wherein R₂ is B(OH)₂.

56. The compound of any one of claims 48-55, wherein R₃ is (C₁-C₆)alkyl.

57. The compound of any one of claims 48-55, wherein R₃ is H.

58. The compound of any one of claims 48-57, wherein R₄ is (C₁-C₆)alkyl.

59. The compound of any one of claims 48-57, wherein R₄ is halo (e.g., fluorine).

60. The compound of any one of claims 48-59, wherein R₅ is O.

61. The compound of any one of claims 48-60, wherein n₂ is 2.

62. The compound of any one of claims 48-60, wherein n₂ is 0.

63. The compound of any one of claims 1-62, wherein Z is ¹⁸F or ¹⁹F.

64. The compound of any one of claims 1-62, wherein Z is ¹³¹I, ¹²³I, ¹²⁴I, or ¹²⁵I.

65. The compound of any one of claims 1-62, wherein Z is ⁷⁶Br or ⁷⁵Br.

66. The compound of any one of claims 1-62, wherein Z is trialkylammonium.

67. The compound of claim 66, wherein Z is N(R₇)₃; and each R₇ is (C₁-C₆)alkyl (e.g., methyl or ethyl).

68. The compound of any one of claims 26-67, wherein L is a bond.

69. The compound of any one of claims 26-67, wherein each L and L₁ is a bond.

70. The compound of any one of claims 1-68, wherein A is a monocyclic cycloalkyl, monocyclic aryl, monocyclic heteroaryl, or monocyclic heterocyclyl.

71. The compound of any one of claims 1-68, wherein each A, A₁, and A₂ is independently a monocyclic cycloalkyl, monocyclic aryl, monocyclic heteroaryl, or monocyclic heterocyclyl.

72. The compound of any one of claims 1-68, wherein A is a bicyclic cycloalkyl, monocyclic aryl, bicyclic heteroaryl, or bicyclic heterocyclyl.

73. The compound of any one of claims 1-68, wherein each A, A₁, and A₂ is independently a bicyclic cycloalkyl, monocyclic aryl, bicyclic heteroaryl, or bicyclic heterocyclyl.

74. The compound of claim 72, wherein the bicycle is fused, bridged, or spirocyclic.

75. The compound of any one of claims 1-68, wherein A is a polycyclic cycloalkyl, polycyclic aryl, polycyclic heteroaryl, or polycyclic heterocyclyl.

76. The compound of any one of claims 1-68, wherein each A, A₁, and A₂ is independently a polycyclic cycloalkyl, polycyclic aryl, polycyclic heteroaryl, or polycyclic heterocyclyl.

77. The compound of any one of claims 1-68, wherein A is heteroaryl.

78. The compound of any one of claims 1-68, wherein each A, A₁, and A₂ is independently heteroaryl.

79. The compound of any one of claims 1-77, wherein A comprises 4-12 ring atoms.

80. The compound of any one of claims 1-77, wherein each A, A₁, and A₂ independently comprises 4-12 ring atoms.

81. The compound of any one of claims 1-79, wherein A comprises 5-12 ring atoms.

82. The compound of any one of claims 1-79, wherein each A, A₁, and A₂ independently comprises 5-12 ring atoms.

83. The compound of any one of claims 1-81, wherein A comprises 6-12 ring atoms.

84. The compound of any one of claims 1-81, wherein each A, A₁, and A₂ independently comprises 6-12 ring atoms.

85. The compound of any one of claims 1-83 wherein A is aryl.

86. The compound of any one of claims 1-83 wherein each A, A₁, and A₂ is independently aryl.

87. The compound of any one of claims 1-83, wherein A further comprises 1-4 hetero ring atoms selected from the group consisting of N, O, and S.

88. The compound of any one of claims 1-83, wherein each A, A₁, and A₂ further independently comprises 1-4 hetero ring atoms selected from the group consisting of N, O, and S.

89. The compound of any one of claims 1-80, wherein A is selected from the group consisting of pentalene, indene, naphthalene, azulene, hetalene, biphenylene, indacene, acenaphthylene, fluorene, phenalene, phenanthrene, anthracene, fluoranthene, acephenanthrylene, aceanthrylene, triphenylene, pyrne, chrysene, naphthacene, pleiadene, picene and perylene.

90. The compound of any one of claims 1-80, wherein each A, A₁, and A₂ is independently selected from the group consisting of pentalene, indene, naphthalene, azulene, hetalene, biphenylene, indacene, acenaphthylene, fluorene, phenalene, phenanthrene, anthracene, fluoranthene, acephenanthrylene, aceanthrylene, triphenylene, pyrne, chrysene, naphthacene, pleiadene, picene and perylene.

91. The compound of any one of claims 1-80, wherein A is selected from the group consisting of azepine, benzofuran, benzopyran, benzothine, chromene, cinnoline, diazepine, diazepinepyrrolopyridine, dioxin, furan, furazan, imidazole, imidazothiazole, indazole, indole, isobenzofuran, isoindole, isopyrazole, isoquinoline, isothianaphthelene, isothiazole, naphthyridine, oxadiazole, oxatriazole, oxazole, oxepin, phthalazine, pteridine, purine, pyran, pyrazine ring, pyrazole, pyridazine, pyridine, pyrimidine, pyrrole, quinazoline, quinolizine, quinoxaline, tetrazole, thaidiazole, thianapthalene, thiazole, thienopyrrole, thiepin, thiophene, ring, and triazole.

92. The compound of any one of claims 1-80, wherein each A, A₁, and A₂ is independently selected from the group consisting of azepine, benzofuran, benzopyran, benzothine, chromene, cinnoline, diazepine, diazepinepyrrolopyridine, dioxin, furan, furazan, imidazole, imidazothiazole, indazole, indole, isobenzofuran, isoindole, isopyrazole, isoquinoline, isothianaphthelene, isothiazole, naphthyridine, oxadiazole, oxatriazole, oxazole, oxepin, phthalazine, pteridine, purine, pyran, pyrazine ring, pyrazole, pyridazine, pyridine, pyrimidine, pyrrole, quinazoline, quinolizine, quinoxaline, tetrazole, thaidiazole, thianapthalene, thiazole, thienopyrrole, thiepin, thiophene, ring, and triazole.

93. The compound of any one of claims 1-80, wherein A is selected from the group consisting of pyridine, quinolone, and isoindole.

94. The compound of any one of claims 1-80, wherein each A, A₁, and A₂ is independently selected from the group consisting of pyridine, quinolone, and isoindole.

95. The compound of any one of claims 1-80, wherein A is selected from the group consisting of phenyl and naphthalene.

96. The compound of any one of claims 1-80, wherein each A, A₁, and A₂ is independently selected from the group consisting of phenyl and naphthalene.

97. The compound of any one of claims 1-80, wherein A selected from the group 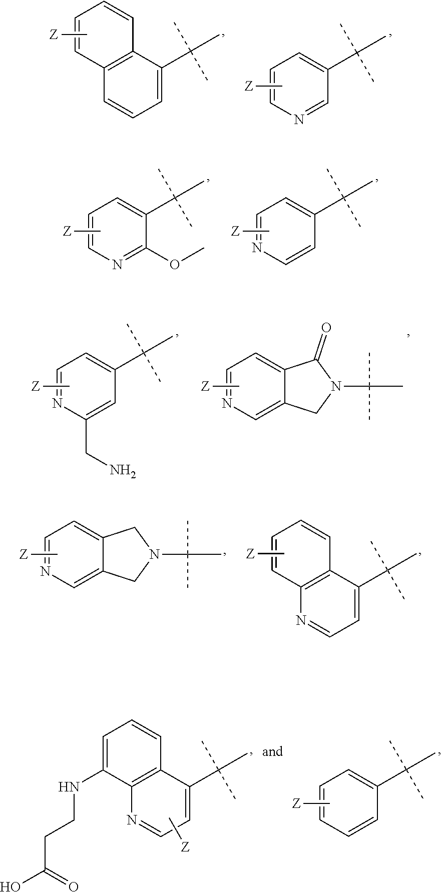
or a pharmaceutically acceptable salt thereof;
Z is a radioactive halogen isotope or a trialkylammonium; and [symbol]
represents the connection to FAPx.

98. The compound of any one of claims 1-80, wherein A is selected from the group consisting of: 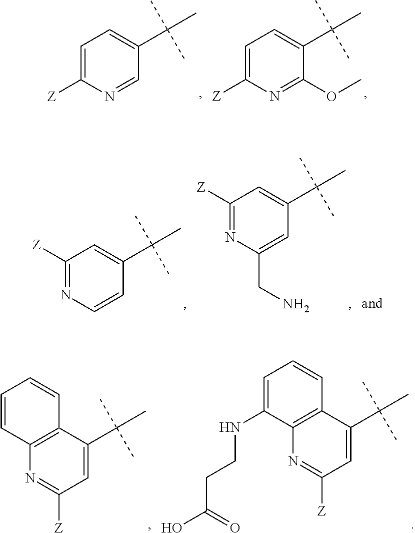
or a pharmaceutically acceptable salt thereof;
Z is a radioactive halogen isotope or a trialkylammonium; and [symbol]
represents the connection to FAPx.

99. The compound of any one of claims 1-80, wherein A is selected from the group consisting of: 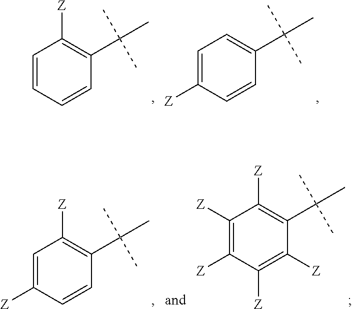
Z is a radioactive halogen isotope or a trialkylammonium; and [symbol]
represents the connection to FAPx.

100. The compound of any one of claims 1-80, wherein A selected from the group consisting of: 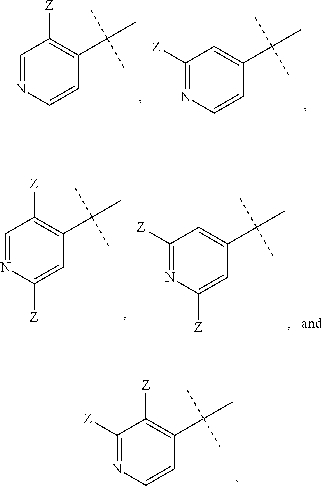
or a pharmaceutically acceptable salt thereof;
Z is a radioactive halogen isotope or a trialkylammonium; and [symbol]
represents the connection to FAPx.

101. The compound of any one of claims 49-100, wherein R₆ is a moiety which modifies the serum half-life of the compound or the tumor distribution of the compound.

102. The compound of any one of claims 49-101, wherein R₆ is a non-proteinaceous half-life extending moiety (e.g., a water soluble polymer such as polyethylene glycol (PEG) or discrete PEG, hydroxyethyl starch (HES)), a lipid, a branched or unbranched acyl group, a branched or unbranched C8-C30 acyl group, a branched or unbranched alkyl group, and a branched or unbranched C8-C30 alkyl group).

103. The compound of any one of claims 49-101, wherein R₆ is a proteinaceous half-life extending moiety (e.g., serum albumin, transferrin, an adnectin (e.g., albumin-binding or pharmacokinetics extending (PKE) adnectins), Fc domain unstructured polypeptide (e.g., XTEN polypeptide, PAS polypeptide, conformationally disordered polypeptide sequences composed of the amino acids Pro, Ala, and/or Ser, or a fragment of any of the foregoing).

104. The compound of any one of claims 1-25, wherein the compound is selected from the group consisting of: 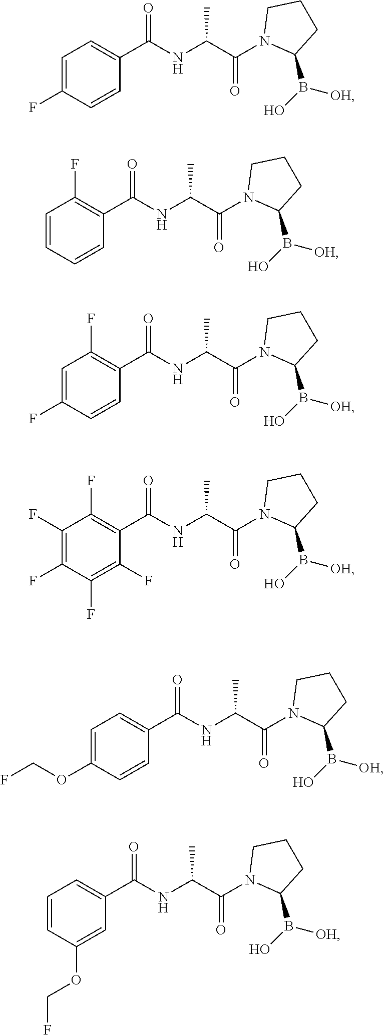 [SMILES: C[C@@H](NC(=O)c1ccc(F)cc1)C(=O)N1CCC[C@H]1B(O)O ; C[C@@H](NC(=O)c1ccccc1F)C(=O)N1CCC[C@H]1B(O)O ; C[C@@H](NC(=O)c1ccc(F)cc1F)C(=O)N1CCC[C@H]1B(O)O ; C[C@@H](NC(=O)c1c(F)c(F)c(F)c(F)c1F)C(=O)N1CCC[C@H]1B(O)O ; C[C@@H](NC(=O)c1ccc(OCF)cc1)C(=O)N1CCC[C@H]1B(O)O ; C[C@@H](NC(=O)c1cccc(OCF)c1)C(=O)N1CCC[C@H]1B(O)O] 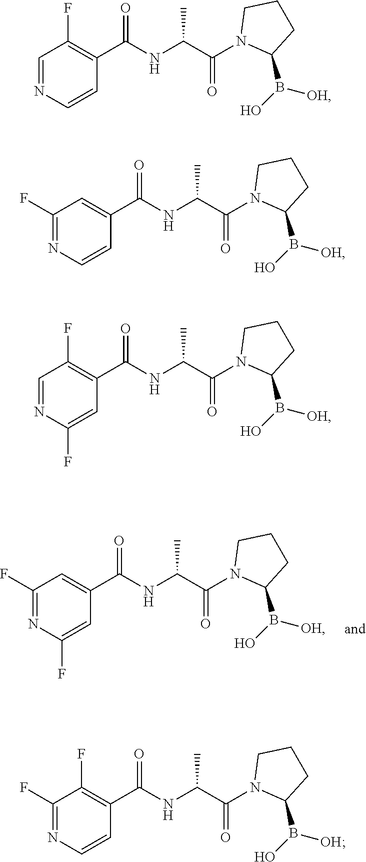 [SMILES: C[C@@H](NC(=O)c1ccncc1F)C(=O)N1CCC[C@H]1B(O)O ; C[C@@H](NC(=O)c1ccnc(F)c1)C(=O)N1CCC[C@H]1B(O)O ; C[C@@H](NC(=O)c1cc(F)ncc1F)C(=O)N1CCC[C@H]1B(O)O ; C[C@@H](NC(=O)c1cc(F)nc(F)c1)C(=O)N1CCC[C@H]1B(O)O ; C[C@@H](NC(=O)c1ccnc(F)c1F)C(=O)N1CCC[C@H]1B(O)O]
or a pharmaceutically acceptable salt thereof.

105. The compound of claim 27, wherein the compound has a structure represented by formula (IV-B-1) or a pharmaceutically acceptable salt thereof: 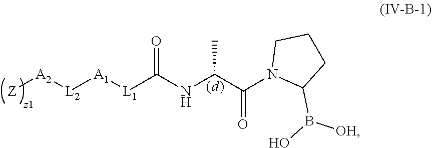
wherein:
A₁ is selected from the group consisting of, 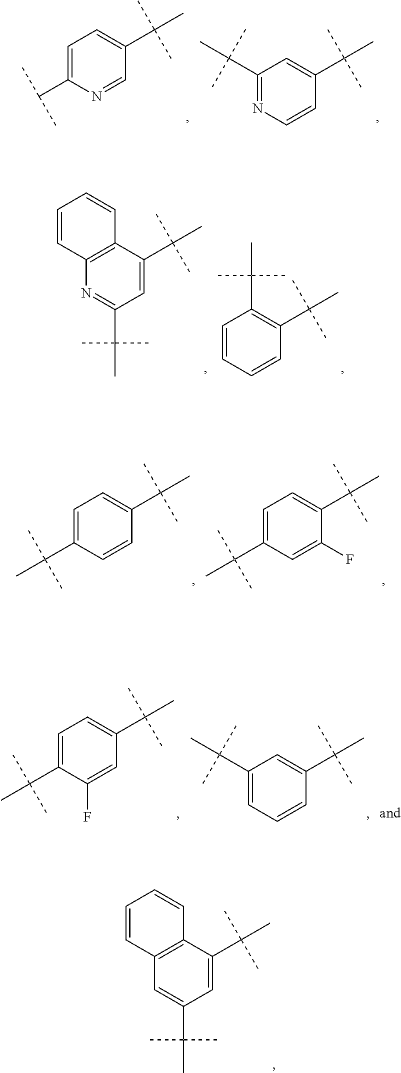
wherein [symbol] represents independently the connection to FAPx and to L₂; and
A₂ is selected from the group consisting of 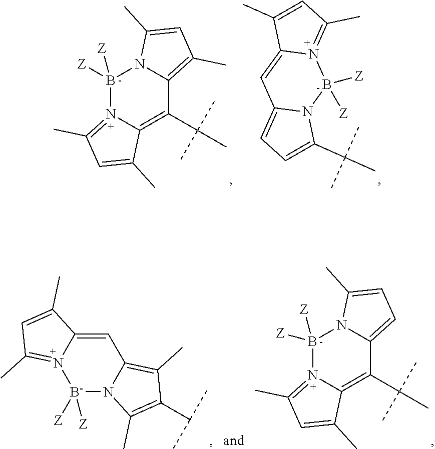
wherein [symbol] represents the connection to L₂; and
each Z is independently a radioactive halogen isotope or a trialkylammonium.

106. The compound of claim 28, wherein the compound has a structure represented by formula (TV-C-1) or a pharmaceutically acceptable salt thereof; 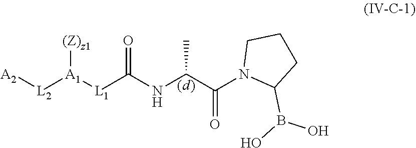
wherein,
A₁ is selected from the group consisting of 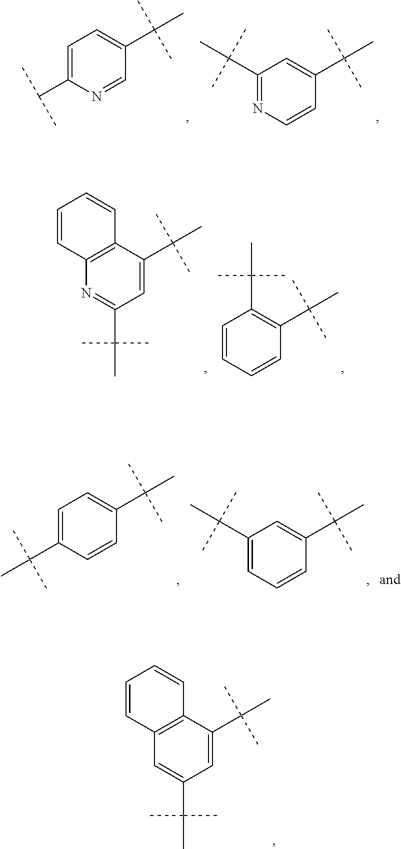
wherein [symbol] represents independently the connection to FAN and to L₂; and
A₂ is selected from the group consisting of 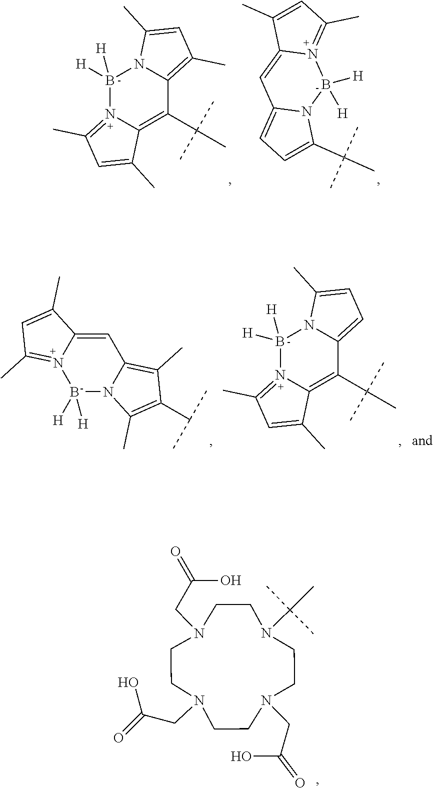
wherein [symbol] represents the connection to L₂; and
each Z is independently a radioactive halogen isotope or a trialkylammonium.

107. A pharmaceutical composition comprising a compound of any one of claims 1-106 and a pharmaceutically acceptable excipient.

108. A method of detecting a cell in a subject, comprising the steps of:
administering to the subject an effective amount of a compound of any one of claims 1-107 or a pharmaceutically acceptable salt thereof; and
obtaining an image of the subject.

109. The method of claim 108, wherein the cell overexpresses FAP.

110. The method of claim 108 or 109, wherein the cell is a cancer cell (e.g., a prostate, pancreatic, colon, or breast cancer cell).

111. The method of any one of claims 108-110, wherein the image is obtained using a positron emission tomography scanner.

112. A method of treating cancer, comprising administering to a subject in need thereof a therapeutically effective amount of a compound of any one of claims 1-108 or a pharmaceutically acceptable salt thereof.

113. The method of claim 112, wherein the cancer is prostate cancer, pancreatic cancer, colon cancer, or breast cancer.

114. A method of making a compound of any one of claims 63-65 and 67-104 wherein Z is a radioactive halogen isotope and A is aryl or heteroaryl, comprising contacting a compound of any one of claims 66-104 wherein Z is a trialkylammonium and A is aryl or heteroaryl with the radioactive halogen isotope, thereby making the compound of any one of claims 63-65 and 67-104 wherein Z is a radioactive halogen isotope and A is aryl or heteroaryl.

115. A method of making a compound of any one of claims 63-65 and 67-104 wherein Z is a radioactive halogen isotope and each A, A₁, and A₂ is independently aryl or heteroaryl, comprising contacting a compound of any one of claims 66-104 wherein Z is a trialkylammonium and each A, A₁, and A₂ is independently aryl or heteroaryl with the radioactive halogen isotope, thereby making the compound of any one of claims 63-65 and 67-104 wherein Z is a radioactive halogen isotope and each A, A₁, and A₂ is independently aryl or heteroaryl.

116. The method of claim 114 or 115, wherein the radioactive halogen isotope is ¹⁸F or ¹⁹F.

117. The method of claim 114 or 115, wherein the radioactive halogen isotope is ¹³¹I, ¹²³I, ¹²⁴I, or ¹²⁵I.

118. The method of claim 114 or 115, wherein the radioactive halogen isotope is ⁷⁶Br or ⁷⁵Br.

119. The method of any one of claims 114-118, wherein the trialkylammonium is trimethylammonium.

120. The method of any one of claims 114-119, wherein the method is performed at about 30° C. to about 70° C.

121. The method of any one of claims 114-119, wherein the method is performed at about 50° C.

122. The method of any one of claims 114-119, wherein the method is performed for about 20 seconds to about 10 minutes.

123. The method of any one of claims 114-122, wherein the method is performed for about 150 seconds.

124. The method of any one of claims 114-122, wherein the method is performed for about 200 seconds.

125. The method of any one of claims 114-122, wherein the method is performed for about 6 minutes.

126. The method of any one of claims 114-125, wherein the method is performed in a microwave.

127. The method of claim 126, wherein the power of the microwave radiation is about 40 to about 60 watts.

128. The method of claim 126 or 127, wherein the power of the microwave radiation is about 50 watts.

129. A kit, comprising a compound of any one of claims 66-96, wherein Z is a trialkylammonium; and instructions for performing the method of any one of claims 125-128.

130. A kit, comprising a compound of any one of claims 74-78 and 81-104, wherein Z is a radioactive halogen isotope; and instructions for performing the method of any one of claims 108-116.
